SEQUENCE LISTING (37 sequences)

SEQ ID NO: 1 - protein, 274 aa, homo sapiens
EPHSLRYNLTVLSWDGSVQSGFLTEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETKEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPS

SEQ ID NO: 2 - protein, 274 aa, homo sapiens
EPHSLRYNLTVLSWDGSVQSGFLAEVHLDGQPFLRYDRQKCRAKPQGQWAEDVLGNKTWDRETRDLTGNGKDLRMTLAHIKDQKEGLHSLQEIRVCEIHEDNSTRSSQHFYYDGELFLSQNVETEEWTVPQSSRAQTLAMNVRNFLKEDAMKTKTHYHAMHADCLQELRRYLESSVVLRRRVPPMVNVTRSEASEGNITVTCRASSFYPRNITLTWRQDGVSLSHDTQQWGDVLPDGNGTYQTWVATRICQGEEQRFTCYMEHSGNHSTHPVPS

SEQ ID NO: 3 - protein, 274 aa, homo sapiens
EPHSLRYNLTVLSWDGSVQSGFLAEVHLDGQPFLRCDRQKCRAKPQGQWAEDVLGNKTWDRETRDLTGNGKDLRMTLAHIKDQKEGLHSLQEIRVCEIHEDNSTRSSQHFYYDGELFLSQNLETEEWTMPQSSRAQTLAMNVRNFLKEDAMKTKTHYHAMHADCLQELRRYLKSGVVLRRTVPPMVNVTRSEASEGNITVTCRASGFYPWNITLSWRQDGVSLSHDTQQWGDVLPDGNGTYQTWVATRICQGEEQRFTCYMEHSGNHSTHPVPS

SEQ ID NO: 4 - protein, 274 aa, homo sapiens
EPHSLRYNLTVLSWDGSVQSGFLAEVHLDGQPFLRYDRQKCRAKPQGQWAEDVLGNKTWDRETRDLTGNGKDLRMTLAHIKDQKEGLHSLQEIRVCEIHEDNSTRSSQHFYYDGELFLSQNLETEEWTVPQSSRAQTLAMNVRNFLKEDAMKTKTHYHAMHADCLQELRRYLESGVVLRRTVPPMVNVTRSEASEGNITVTCRASSFYPRNIILTWRQDGVSLSHDTQQWGDVLPDGNGTYQTWVATRICRGEEQRFTCYMEHSGNHSTHPVPS

SEQ ID NO: 5 - protein, 274 aa, homo sapiens
EPHSLRYNLTVLSWDGSVQSGFLAEVHLDGQPFLRYDRQKCRAKPQGQWAEDVLGNKTWDRETRDLTGNGKDLRMTLAHIKDQKEGLHSLQEIRVCEIHEDNSTRSSQHFYYDGELFLSQNLETEEWTVPQSSRAQTLAMNVRNFLKEDAMKTKTHYHAMHADCLQELRRYLESSVVLRRTVPPMVNVTRSEASEGNITVTCRASSFYPRNIILTWRQDGVSLSHDTQQWGDVLPDGNGTYQTWVATRICRGEEQRFTCYMEHSGNHSTHPVPS

SEQ ID NO: 6 - protein, 115 aa, Artificial
  Chimeric
QVQLQESGPGLVKPSETLSLTCTVSGYSITSDYAWNWIRQPPGKGLEWIGFVSYSGTTKYNPSLKSRVTISRDTSKNQFSLKLSSVTAADTAVYYCARGYGFDYWGQGTTVTVSS

SEQ ID NO: 7 - protein, 108 aa, artificial
  Chimeric homo sapiens-mus musculus
EIVLTQSPATLSLSPGERATLSCSATSSISSIYFHWYQQKPGQAPRLLIYRTSNLASGIPARFSGSGSGTDYTLTISSLEPEDFAVYYCQQGTTIPFTFGQGTKLEIK

SEQ ID NO: 8 - protein, 115 aa, artificial; identical to SEQ ID NO: 6
  Chimeric homo sapiens-mus musculus

SEQ ID NO: 9 - protein, 108 aa, artificial
  Chimeric homo sapiens-mus musculus
EIVLTQSPATLSLSPGERATLSCSATSSISSIYFHWYQQKPGQAPRLLIYRTSNLASGIPARFSGSGSGTSYTLTISSLEPEDFAVYYCQQGTTIPFTFGQGTKLEIK

SEQ ID NO: 10 - protein, 115 aa, artificial; identical to SEQ ID NO: 6
  Chimeric homo sapiens-mus musculus

SEQ ID NO: 11 - protein, 108 aa, artificial
  Chimeric homo sapiens-mus musculus
EIVLTQSPATLSLSPGERATLSCSATSSISSIYFHWYQQKPGQAPRLLIYRTSNLASGIPARFSGSGSGTDYTLTISSLEPEDVAVYYCQQGTTIPFTFGQGTKLEIK

SEQ ID NO: 12 - protein, 115 aa, artificial
  Chimeric homo sapiens-mus musculus
QVQLQESGPGLVKPSETLSLTCTVSGYSITSDYAWNWIRQPPGKGLEWMGFVSYSGTTKYNPSLKSRVTISRDTSKNQFSLKLSSVTAADTAVYYCARGYGFDYWGQGTTVTVSS

SEQ ID NO: 13 - protein, 108 aa, artificial; identical to SEQ ID NO: 7
  Chimeric homo sapiens-mus musculus

SEQ ID NO: 14 - protein, 115 aa, artificial; identical to SEQ ID NO: 12
  Chimeric homo sapiens-mus musculus

SEQ ID NO: 15 - protein, 108 aa, artificial; identical to SEQ ID NO: 9
  Chimeric homo sapiens-mus musculus

SEQ ID NO: 16 - protein, 115 aa, artificial; identical to SEQ ID NO: 12
  Chimeric homo sapiens-mus musculus

SEQ ID NO: 17 - protein, 108 aa, artificial; identical to SEQ ID NO: 11
  Chimeric homo sapiens-mus musculus

SEQ ID NO: 18 - protein, 115 aa, artificial
  Chimeric homo sapiens-mus musculus
QVQLQESGPGLVKPSETLSLTCTVSGYSITSDYAWNWIRQPPGKGLEWMGFVSYSGTTKYNPSLKSRVTISRDTSENQFSLKLSSVTAADTAVYYCARGYGFDYWGQGTTVTVSS

SEQ ID NO: 19 - protein, 108 aa, artificial; identical to SEQ ID NO: 7
  Chimeric homo sapiens-mus musculus

SEQ ID NO: 20 - protein, 115 aa, artificial; identical to SEQ ID NO: 18
  Chimeric homo sapiens-mus musculus

SEQ ID NO: 21 - protein, 108 aa, artificial; identical to SEQ ID NO: 9
  Chimeric homo sapiens-mus musculus

SEQ ID NO: 22 - protein, 115 aa, artificial; identical to SEQ ID NO: 18
  Chimeric homo sapiens-mus musculus

SEQ ID NO: 23 - protein, 108 aa, artificial; identical to SEQ ID NO: 11
  Chimeric homo sapiens-mus musculus

SEQ ID NO: 24 - protein, 115 aa, artificial
  Chimeric homo sapiens-mus musculus
QVQLQESGPGLVKPSETLSLTCTVSGYSITSDYAWNWIRQPPGKGLEWMGFVSYSGTTKYNPSLKSRITISRDTSENQFSLKLSSVTAADTAVYYCARGYGFDYWGQGTTVTVSS

SEQ ID NO: 25 - protein, 108 aa, artificial; identical to SEQ ID NO: 7
  Chimeric homo sapiens-mus musculus

SEQ ID NO: 26 - protein, 115 aa, artificial; identical to SEQ ID NO: 24
  Chimeric homo sapiens-mus musculus

SEQ ID NO: 27 - protein, 108 aa, artificial; identical to SEQ ID NO: 9
  Chimeric homo sapiens-mus musculus

SEQ ID NO: 28 - protein, 115 aa, artificial; identical to SEQ ID NO: 24
  Chimeric homo sapiens-mus musculus

SEQ ID NO: 29 - protein, 108 aa, artificial; identical to SEQ ID NO: 11
  Chimeric homo sapiens-mus musculus

SEQ ID NO: 30 - protein, 6 aa, mus musculus
SDYAWN

SEQ ID NO: 31 - protein, 16 aa, mus musculus
FVSYSGTTKYNPSLKS

SEQ ID NO: 32 - protein, 6 aa, mus musculus
GYGFDY

SEQ ID NO: 33 - protein, 12 aa, mus musculus
SATSSISSIYFH

SEQ ID NO: 34 - protein, 6 aa, mus musculus
RTSNLA

SEQ ID NO: 35 - protein, 9 aa, mus musculus
QQGTTIPFT

SEQ ID NO: 36 - protein, 383 aa, homo sapiens [sequence omitted]

SEQ ID NO: 37 - protein, 218 aa, homo sapiens
PAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2017/0267764 A1
(43) Pub. Date: Sep. 21, 2017

(54) ANTI-MICA ANTIBODIES

(71) Applicant: INNATE PHARMA, MARSEILLE (FR)

(72) Inventors: MATHIEU BLERY, MARSEILLE (FR); LAURENT GAUTHIER, MARSEILLE (FR)

(21) Appl. No.: 15/458,123

(22) Filed: Mar. 14, 2017

Related U.S. Application Data
(60) Provisional application No. 62/308,443, filed on Mar. 15, 2016.

(51) Int. Cl. C07K 16/28; C12Q 1/68; G01N 33/574

(52) U.S. Cl. CPC C07K 16/2833; G01N 33/57492; C12Q 1/6886; C07K 2317/56; C07K 2317/567; C07K 2317/565; C07K 2317/76; C07K 2317/92; C07K 2317/52; C12Q 2600/158; G01N 2333/70539; A61K 2039/505

(57) ABSTRACT

The present invention provides antigen-binding proteins capable of binding to human MICA polypeptides. The antigen-binding proteins have increased activity in the treatment of disorders characterized by MICA-expressing cells, particularly cancer.

### CROSS-REFERENCE TO RELATED APPLICATION

This application claims the benefit of U.S. Provisional Application No. 62/308,443 filed Mar. 15, 2016, which is incorporated herein by reference in its entirety; including any drawings and sequence listing.

### REFERENCE TO THE SEQUENCE LISTING

The present application is being filed along with a Sequence Listing in electronic format. The Sequence Listing is provided as a file entitled “MICA2-PCT_ST25.txt”, created Mar. 13, 2017, which is 44 KB in size. The information in the electronic format of the Sequence Listing is incorporated herein by reference in its entirety.

### FIELD OF THE INVENTION

The present invention provides antigen-binding proteins capable of binding to MICA polypeptides. The antigen-binding proteins have increased activity in the treatment of disorders characterized by MICA-expressing cells, particularly cancer.

### BACKGROUND

The immunoreceptor NKG2D is normally expressed on human T cells (e.g., CD8⁺ T cells, γδ T cells) and NK cells. On pre-activated CD8⁺ cells, NKG2D functions as a synergistic co-stimulator of CD28 and TCR signalling via DAP10 association, whereas in NK cells it functions as a direct activator. Upon ligand engagement, NKG2D therefore conveys directly activating or costimulatory signals via the paired DAP10 adaptor protein, thereby promoting cancer and infectious disease immunity.

Various ligands for human NKG2D (hNKG2D) have been identified and characterized, including the major histocompatibility complex class I-related chain A and B polypeptides (MICA and MICB), the UL16-binding protein (ULBP) family, and the retinoic acid early transcript-1 (RAET1) family. MICA is frequently associated with epithelial tumors, induced by microbial infections, and aberrantly expressed in certain autoimmune disease lesions. The structure of MICA is similar to the protein fold of MHC class I, with an α 1α2 platform domain and a membrane-proximal Ig-like α3 domain (Li et al 2001 Nat. Immunol. 2:443). MICA and its close relative MICB, which also serves as a ligand for NKG2D, are both polymorphic and the polymorphism has been shown to affect the affinity for NKG2D (Steinle et al. 2001 Immunogenetics 53:279).

In the mouse, which lacks MHC class I chain (MIC) genes, a family of proteins structurally related to ULBP, the retinoic acid early (RAE-1) molecules function as ligands for NKG2D. RAE-1 expression has been shown to be induced by carcinogens and to stimulate antitumor activities of T cells. Murine NKG2D, however, recognizes human MICA polypeptides (Wiemann (2005) J. Immunol. 175:820-829).

The role of MICA in cancer biology has been complicated by the fact that MICA is released as a soluble form from the cell surface of tumor cells (e.g., *019 allele) and on the surface of exosomes (*08 allele) (Ashiru et al (2010) Cancer Res. 70(2):481-489)). Soluble MICA (sMICA) can be detected for example at high levels in sera of patients with gastrointestinal malignancies (Salih et al, 2002 J. Immunol. 169: 4098). The MMPs ADAM10 and ADAM17, as well as the disulfide isomerase Erp5, have been reported to have a role in cleavage and shedding of MICA (Waldhauer (2008) Cancer Research 68 (15) 6368-76; Kaiser et al (2007) Nature; and Salih (2002) J. Immunol 169: 4098-4102). Membrane bound MICA has been reported to downmodulate the expression of NKG2D on NK and/or T cells (Von Lilienfeld-Toal et al. (2010) Cancer Immunol. Immunother.). Notably, Wemann (2005), supra, examined MICA Tg mice and concluded that down-regulation of surface NKG2D on nontransgenic splenocytes was most pronounced after cocultivation with splenocytes from MICA transgenic mice in vitro, and only marginally following treatment with sera from H2Kb-MICA mice, whereas incubation with control cells and sera from nontgLM, respectively, had no effect and that overall data suggest that reduced surface NKG2D on H2-K-MICA NK cells results in NKG2D dysfunction and that NKG2D downregulation is primarily caused by a persistent exposure to cellbound MICA in vivo.

Reports have also emerged that NKG2D on NK cells is downregulated by sMICA (Groh et al. (2002) Nature; Arreygue-Garcia (2008) BMC; Jinushi et al. (2005) J. Hepatol.), leading to less reactive NK cells. This rationale may have emerged because similar systems have been observed among other protein families such as the Ig-like and the TNF superfamily have been shown to be released as a soluble form and that release of the molecules affects cell-cell interactions by reduction of ligand densities and modulates NK cells bearing the respective receptor (Salih 2002). Consequently, attempts to generate anti-MICA antibodies have focused on development of antibodies that inhibit MICA shedding.

It has also been reported that expression of NKG2D ligands MICA and MICB on healthy cells can break the balance between immune activation and tolerance, and trigger autoimmunity. Genetic linkage studies have shown that some MICA alleles are positively associated with type 1 diabetes, and development of disease in prediabetic NOD mice expressing Rae1 on their islet cells can be completely prevented by treatment with NKG2D-blocking mAbs, which reduce expansion and function of autoreactive CD8+ T cells. MICA and MICB molecules are also dramatically upregulated in RA synoviocytes and activate the T cells in an NKG2D-dependent manner. Moreover, rheumatoid arthritis patients have been reported to have high levels of IL-15 and TNF-a in the sera and inflamed joints which induce expression of NKG2D on CD4+CD28-subset of T cells. In Celiac disease, massive infiltration of intraepithelial NKG2D+ CD8+ T lymphocytes in the gut has been reported, and MIC proteins become strongly expressed on the surface of epithelial cells in patients with active disease. In inflammatory bowel disorders, increased levels of MIC expression were found on intestinal epithelial cells and it the number of intestinal epithelial CD4+ T cells expressing NKG2D was found to correlate with intestinal inflammation.

Approaches to date to treat inflammation based on the NKG2D system have focused on blockade of NKG2D itself rather than its ligands (Ogasawara et al. (2004) Immunity 20(6):757-767; Andersson et al (2011) Arthritis. Rheum. 63(9):2617-2629; Steigerwald et al (2009) MAbs 1(2):115-127. One possibility is that this focus on NKG2D rather than its ligand is due to the perceived difficulty of targeting the NKG2D ligand system which includes a variety of ligands and in some cases a large number of alleles.

For MICA and MICB, there are over 97 MICA alleles and at least 31 MICB alleles recognized. There is only 43% amino acid identity across the MIC polypeptides in the α1α2 domain (the domain involved in the NKG2D interface), and 80% of the amino acid substitutions are non-conservative (Steinle et al. (2001) Immunogenetics 53: 279-287; Steinle et al. (1998) Proc. Natl. Acad. Sci. U.S.A. 95:12510-12515), suggesting that it will be unlikely to obtain antibodies that are effective for a majority of individuals in a population. Additionally, the methionine/valine bimorphism at position 129 in MICA determines differences in NKG2D binding, and although the side chain of residue 129 is partially buried and forms hydrophobic interactions with glutamine 136, alanine 139 and methionine 140 in the first α2 helical stretch, it may be associated with a difference in conformation in this domain in comparison with valine 129 forms of MICA (Steinle et al (2001) Immunogenetics 53: 279-287).

In conclusion, there is a need for new approaches to target MICA with therapeutic agents.

### SUMMARY OF THE INVENTION

In one aspect, the invention results, inter alia, from the discovery of antibodies with high affinity across human MICA alleles (as well as on non-human primate MICA).

In one embodiment, provided is an anti-MICA antigen binding domain, or a protein that comprises the antigen binding domain (e.g., a monoclonal antibody, a multispecific binding protein, a bispecific antibody, etc.), the antigen binding domain comprising:

(a) a heavy chain variable region (VH) comprising an amino acid sequence at least 80%, 90%, 95% or 98% identical to the amino acid sequence of SEQ ID NO: 6, and

(b) a light chain variable region (VL) comprising an amino acid sequence at least 80%, 90%, 95% or 98% identical to the amino acid sequence of SEQ ID NO: 7.

In one embodiment, the VL comprises a tyrosine (Y) amino acid residue at Abnum position 71 (in FR3). In one embodiment, the VL comprises a phenylalanine (F) at Abnum position 83.

In one embodiment, the heavy chain variable region (VH) comprises amino acid residues at Abnum positions 72c (in FR2) and 74 (in FR3) capable of interacting with one another by H-bonding between the residue at position 72c and the residue at position Abnum 74. In one embodiment, the VH comprises a lysine (K) amino acid residue at Abnum position 72c and a glutamine residue at position 74. In one embodiment, the VH comprises a threonine (T) at Abnum position 30. In one embodiment, the VH comprises an isoleucine (I) at Abnum position 48. In one embodiment the VH comprises a valine (V) at Abnum position 67. In one embodiment, the VH comprises an arginine (R) at Abnum position 71.

In one embodiment, the VH segment of the VH human acceptor framework is from IGHV4-b (e.g., IGHV4-b*02) and the J-segment is from IGHJ6 (e.g., IGHJ6*01). In one embodiment, the CDR1, 2 and 3 of the VH comprise the amino acid sequences of SEQ ID NOS: 30, 31 and 32, respectively. In one embodiment, the VL domain human acceptor framework is from IGKV3-11 (e.g., IGKV3-11*01) and the J-segment is from IGKJ2 (e.g., IGKJ2*01). In one embodiment, the CDR1, 2 and 3 of the VL comprise the amino acid sequences of SEQ ID NOS: 33, 34 and 35, respectively. In one embodiment, the human heavy chain and/or light chain acceptor framework comprises one or more back-mutations in which an amino acid is substituted by an amino acid present at the particular position in a non-human mammal (e.g., murine, rat). In one embodiment, the human heavy chain acceptor framework 1 (FR1) comprises a threonine (T) at Abnum position 30 and contains no other mutations compared to a naturally occurring human VH segment. In one embodiment, the human heavy chain acceptor framework 2 (FR2) is free of mutations compared to a naturally occurring human VH segment. In one embodiment, the human heavy chain acceptor framework 3 (FR3) comprises a arginine (R) at Abnum position 71 and contains no other mutations compared to a naturally occurring human VH segment. In one embodiment, the human heavy chain acceptor framework 4 (FR4) is free of mutations compared to a naturally occurring human VH segment. In one embodiment, the human light chain acceptor framework 3 (FR3) comprises a tyrosine at Abnum position 71 and contains no other mutations compared to a naturally occurring human VH segment. In one embodiment, the human light chain acceptor frameworks 1, 2 and 4 (FR1, FR2 and FR4) are free of mutations compared to a naturally occurring human VH segment.

In one embodiment of any aspect herein, the VH comprises the heavy chain CDR1, CDR2 and CDR3 having the respective amino acid sequences shown in SEQ ID NOS: 30, 31 and 32. In one embodiment, the VL comprises the light chain CDR1, CDR2 and CDR3 having the respective amino acid sequences shown in SEQ ID NOS: 33, 34 and 35.

In one embodiment, provided is an anti-MICA antigen binding domain, or a protein that comprises the antigen binding domain (e.g., a monoclonal antibody, a multispecific binding protein, a bispecific antibody, etc.), comprising:

(a) a heavy chain variable region comprising an amino acid sequence of SEQ ID NO: 6, optionally further comprising one, two or three amino acid residue substitutions in a framework region, and

(b) a light chain variable region comprising an amino acid sequence of SEQ ID NO: 7, optionally further comprising one, two or three amino acid residue substitutions in a framework region.

In one embodiment, provided is an anti-MICA antigen binding domain, or a protein that comprises the antigen binding domain (e.g., a monoclonal antibody, a multispecific binding protein, a bispecific antibody, etc.), comprising:

(a) a heavy chain variable region comprising an amino acid sequence of SEQ ID NO: 8, optionally further comprising one, two or three amino acid residue substitutions in a framework region, and

(b) a light chain variable region comprising an amino acid sequence of SEQ ID NO: 9, optionally further comprising one, two or three amino acid residue substitutions in a framework region.

In one aspect of any of the embodiments, the light chain variable region comprises a tyrosine (Y) residue at position 71 (Abnum numbering).

In another aspect of any of the embodiments, the heavy chain variable region comprises a lysine (K) residue as position 72c (Abnum numbering).

In one embodiment, provided is an anti-MICA antigen binding domain, or a protein that comprises the antigen binding domain (e.g., a monoclonal antibody, a multispecific binding protein, a bispecific antibody, etc.), the antigen binding domain selected from the group consisting of:

(a) an antibody binding domain comprising a heavy chain variable region comprising an amino acid sequence of SEQ ID NO: 6 and a light chain variable region comprising an amino acid sequence of SEQ ID NO: 7;

(b) an antibody binding domain comprising a heavy chain variable region comprising an amino acid sequence of SEQ ID NO: 8 and a light chain variable region comprising an amino acid sequence of SEQ ID NO: 9; and

(c) an antibody binding domain comprising a heavy chain variable region comprising an amino acid sequence of SEQ ID NO: 10 and a light chain variable region comprising an amino acid sequence of SEQ ID NO: 11.

In one embodiment, provided is a monoclonal antibody that binds human MICA, selected from the group consisting of:

(a) an antibody comprising a heavy chain variable region comprising an amino acid sequence of SEQ ID NO: 6 and a light chain variable region comprising an amino acid sequence of SEQ ID NO: 7;

(b) an antibody comprising a heavy chain variable region comprising an amino acid sequence of SEQ ID NO: 8 and a light chain variable region comprising an amino acid sequence of SEQ ID NO: 9; and

(c) an antibody comprising a heavy chain variable region comprising an amino acid sequence of SEQ ID NO: 10 and a light chain variable region comprising an amino acid sequence of SEQ ID NO: 11.

In one aspect, provided are anti-MICA antibodies with human frameworks that have modified salt bridges; salt bridges in proteins are H-bonds between oppositely charged residues that are sufficiently close to each other to experience electrostatic attraction. In one aspect, provided are anti-MICA antibodies with human frameworks that have amino acid substitutions in the light chain FR3. In one aspect, an antibody comprises H-bonding in the heavy chain FR3 region between residues at positions 72c and 74 (Abnum numbering).

The antibodies notably bind to the predominant MICA alleles from each of two major MICA groups that are determined to represent the main families of MICA: Group 1 alleles that bind NKG2D strongly (including MICA*001, *002, *007, *012, *017 and *018) and Group 2 that bind NKG2D weakly (MICA*004, *006, *008, *009 and *019). By binding to an epitope present on the subset MICA *001, *004, *007 and *008 or *001, *004, *007, *008 and *019, the antibodies cover the alleles of both groups that are present in almost all individuals. Optionally, the antibodies have an EC₅₀, as determined by flow cytometry, of no more than 1 μg/ml, optionally no more than 0.5 μg/ml, no more than 0.3 μg/ml, or no more than 0.2 μg/ml for binding to cells made to express at their surface *001, to cells made to express at their surface *004, to cells made to express at their surface *007 and to cells made to express at their surface *008. Optionally, the antibodies have an EC₅₀, as determined by flow cytometry, of no more than 0.1 μg/ml, optionally no more than 0.07 μg/ml for binding to cells made to express at their surface *004, to cells made to express at their surface *007 and to cells made to express at their surface *008. The antibodies optionally further bind to cells expressing a human MICB polypeptide.

In one embodiment, an antibody that is capable of binding MICA alleles has an EC₅₀ for binding to a human MICA*001 that differs by less than 1-log from its binding affinity for human MICA*004, *007 and/or *008, as determined by flow cytometry for binding to cells expressing at their surface the respective MICA polypeptide cells transfected with one of the respective MICA alleles but that do not express the other MICA alleles). In one embodiment, the antibody has an EC₅₀ for binding to human MICA*004, *007 and/or *008 polypeptide that differs from each other by no more than 0.5 log, 0.3 log or 0.2 log, as determined by flow cytometry for binding to cells expressing at their surface human MICA*004, *007 and/or *008.

Optionally, the EC₅₀ is determined according the methods of the Examples herein, or according to Example 3 of PCT publication no WO2013/117647, e.g. C1R cells (ATCC reference CRL-1993™) transfected with RSV.5neo vectors (GenBank (NCBI) under Accession number M83237) containing the MICA nucleic acid of interest, data acquisition by flow cytometry and EC₅₀ computation using a 4 parameter model.

High affinity binding is advantageous, inter alia, for an antibody to effectively mediate CDC and/or ADCC.

The antibodies of the disclosure are capable of blocking the interaction of MICA on the surface of cells (e.g., tumor cells) with NKG2D (e.g., on NK cells and T cells). Thus, in addition to induction of ADCC and/or CDC activity when comprising Fc domains that are bound by Fcγ receptors, these antibodies are useful for their ability to be able to block membrane MICA-induced down-modulation of NKG2D, e.g., for the treatment of cancer and/or infectious disease. Furthermore, in addition to or alternatively to the ability to mediate ADCC and/or CDC activity, these antibodies are useful for their ability to reduce M2 macrophage-mediated suppression of T cell and/or NK cell activity. In other embodiments, antibodies which do not substantially induce ADCC and/or CDC activity (e.g., do not comprise an Fc domain that is bound by FcγIIIa receptors) can be useful for their ability to be able to block membrane MICA-induced down-modulation of NKG2D and/or to reduce M2 macrophage-mediated suppression of T cell and/or NK cell activity, for the treatment of inflammatory and/or autoimmune disorders. In yet further embodiment, the antibodies can be conjugated to a toxic agent (e.g., a cytotoxic moiety) and used to cause the depletion or death of MICA-expressing cells (e.g. tumor cells).

In one aspect, provided are methods of treatment using the anti-MICA antibodies of the invention. The antibodies can be used as prophylactic or therapeutic treatment; in any of the embodiments herein, a therapeutically effective amount of the antibody can be interchanged with a prophylactically effective amount of an antibody. In one aspect, provided is a method of treating an individual with a cancer, an autoimmune disorder or an inflammatory disorder, the method comprising administering to the individual a pharmaceutically effective amount of an antigen-binding compound according to the disclosure that specifically binds to a MICA polypeptide.

In one aspect, provided is a method of eliminating a MICA-expressing cell (e.g. a cancer cell) in an individual, the method comprising administering to the patient a pharmaceutically effective amount of an antigen-binding compound according to the disclosure that specifically binds to a MICA polypeptide. In one aspect, provided is a method of overcoming or reducing myeloid-derived suppression cell (MDSC)-mediated suppression of NK cell and/or T cell activity in an individual having a cancer, the method comprising administering to the individual a pharmaceutically effective amount of an antigen-binding compound according to the disclosure that specifically binds to a MICA polypeptide. In one aspect, provided is a method eliminating or inhibiting the immunosuppressive activity of myeloid-derived suppression cells (MDSC) and/or M2 macrophages, e.g., tumor tissue resident MDSC or M2 cells, in an individual having a cancer, the method comprising administering to the individual a pharmaceutically effective amount of an antigen-binding compound according to the disclosure that specifically binds to a MICA polypeptide.

In another aspect, provided is a method (e.g., a method of conducting a diagnostic assay, a responder assay, etc.), comprising assessing whether a patient has disease-related cells (e.g., tumor cells) expressing a MICA polypeptide, e.g., a MICA polypeptide (one or more MICA alleles) bound by an antibody of the disclosure. Said method may comprise, for example, obtaining a biological sample from a patient comprising disease-related cells, bringing said disease-related cells into contact with such antibody and assessing whether the antibody binds to disease-related cells. A finding that MICA is expressed by disease-related cells indicates that the patient has a condition characterized by MICA-expressing cells and/or is suitable for treatment with an anti-MICA antibody of the disclosure. The patient can further be treated with a treatment suitable for the particular disease characterized by MICA-expressing cells. Optionally the patient is treated with the anti-MICA antibody. In one embodiment, the method is used for selecting subjects having a cancer, and the disease-related cells are cancer cells.

These aspects are more fully described in, and additional aspects, features, and advantages will be apparent from, the description provided herein.

### BRIEF DESCRIPTION OF THE DRAWINGS

FIG. 1 shows that anti-MICA mAb1 induced specific lysis of C1R-MICA*001 and *008 cells by human KHYG-1 CD16-expressing NK cell compared to negative controls (Human IgG1 isotype control antibody) and to its parental (unmodified) chimeric antibody, thereby showing that these antibodies induce ADCC toward MICA*001 and *008-expressing target cells.

FIG. 2 shows that anti-MICA mAb1 caused a strong increase in NK cell activation towards the 721.221-MICA*001 tumor cells, with or without M1 or M2 macrophages. In contrast, in isotype control, not only was NK activation generally far lower, but incubation of tumor cells and NK cells with M2 macrophages caused a strong decrease in NK activation.

FIG. 3 shows that while mice receiving isotype control or 1 μg anti-MICA antibody mAb1 did not survive at 100 days post injection, significantly improved survival was observed in mice receiving at least 10 μg of anti-MICA antibody. At the 100 μg dose, anti-MICA antibody mAb1 achieved survival in all mice at 100 days.

FIG. 4 shows, in the left hand panel, mice receiving isotype control, and in the right hand panel, mice receiving anti-MICA antibody mAb1. Individual tumor volumes are shown. CR=complete response. Treatment with anti-MICA antibody mAb1 caused a decrease in tumor volume.

FIG. 5 shows that mice treated with anti-MICA antibody mAb1 exhibited a decreased tumor cell count compared to mice treated with isotype control.

### DETAILED DESCRIPTION OF THE INVENTION

The antibodies of the invention are able to directly and specifically target MICA-expressing cells as well as MICB-expressing cells, notably tumor cells and cells involved in inflammatory or autoimmune processes.

MICA (PERB11.1) refer to MHC class I polypeptide-related sequence A (See, e.g., UniProtKB/Swiss-Prot Q29983), its gene and cDNA and its gene product, or naturally occurring variants thereof. Nomenclature of MICA genes and proteins, together with reference to accession number of sequence for different alleles are described in Frigoul A. and Lefranc, M-P. Recent Res. Devel. Human Genet., 3(2005): 95-145 ISBN: 81-7736-244-5, the disclosure of which is incorporated herein by reference. MICA genes and protein sequence, including polymorphisms at the protein and DNA level, are also available from http://www.ebi.ac.uk/ipd/imgt/hla/align.html maintained by Cancer Research UK and the European Bioinformatics Institute (EBI).

The amino acid sequences of MICA were first described in Bahram et al (1994) Proc. Nat. Acad. Sci. 91: 6259-6263 and Bahram et al. (1996) Immunogenetics 44:80-81, the disclosures of which are incorporated herein by reference. The MICA gene is polymorphic, displaying an unusual distribution of a number of variant amino acids in their extracellular α1, α2, and α3 domains. To further define the polymorphism of MICA, Petersdorf et al. (1999) examined its alleles among 275 individuals with common and rare HLA genotypes. The amino acid sequence of the extracellular α1, α2, and α3 domains of human MICA are shown in SEQ ID NOS: 1-5. The full MICA sequence further comprises a leader sequence of 23 amino acids, as well as a transmembrane domain and a cytoplasmic domain. The amino acid sequence of extracellular α1, α2, and α3 domains of selected human MICA alleles are shown in SEQ ID NOS: 1-5. The amino acid sequence of MICA*001 is shown in SEQ ID NO: 1, corresponding to Genbank accession no. AAB41060. The amino acid sequence of human MICA allele MICA*004 is shown in SEQ ID NO: 2, corresponding to Genbank accession no. AAB41063. The amino acid sequence of human MICA allele MICA*007 is shown in SEQ ID NO: 3, corresponding to Genbank accession no. AAB41066. The amino acid sequence of human MICA allele MICA*008 is shown in SEQ ID NO: 4, corresponding to Genbank accession no. AAB41067. The amino acid sequence of human MICA allele MICA*019 is shown in SEQ ID NO: 5, corresponding to Genbank accession no. AAD27008. The amino acid sequence of human MICB is shown Genbank accession no. CA118747 (SEQ ID NO: 36).

| MICA | | |
|---|---|---|
| Allele | SEQ ID | Amino acid sequence |
| MICA*001 | 1 | EPHSLRYNLT VLSWDGSVQS GFLTEVHLDG QPFLRCDRQK CRAKPQGQWA |
| | | EDVLGNKTWD RETRDLTGNG KDLRMTLAHI KDQKEGLHSL QEIRVCEIHE |
| | | DNSTRSSQHF YYDGELFLSQ NLETKEWTMP QSSRAQTLAM NVRNFLKEDA |
| | | MKTKTHYHAM HADCLQELRR YLKSGVVLRR TVPPMVNVTR SEASEGNITV |
| | | TCRASGFYPW NITLSWRQDG VSLSHDTQQW GDVLPDGNGT YQTWVATRIC |
| | | QGEEQRFTCY MEHSGNHSTH PVPS |
| MICA*004 | 2 | EPHSLRYNLT VLSWDGSVQS GFLAEVHLDG QPFLRYDRQK CRAKPQGQWA |
| | | EDVLGNKTWD RETRDLTGNG KDLRMTLAHI KDQKEGLHSL QEIRVCEIHE |
| | | DNSTRSSQHF YYDGELFLSQ NVETEEWTVP QSSRAQTLAM NVRNFLKEDA |
| | | MKTKTHYHAM HADCLQELRR YLESSVVLRR RVPPMVNVTR SEASEGNITV |
| | | TCRASSFYPR NITLTWRQDG VSLSHDTQQW GDVLPDGNGT YQTWVATRIC |
| | | QGEEQRFTCY MEHSGNHSTH PVPS |
| MICA*007 | 3 | EPHSLRYNLT VLSWDGSVQS GFLAEVHLDG QPFLRCDRQK CRAKPQGQWA |
| | | EDVLGNKTWD RETRDLTGNG KDLRMTLAHI KDQKEGLHSL QEIRVCEIHE |
| | | DNSTRSSQHF YYDGELFLSQ NLETEEWTMP QSSRAQTLAM NVRNFLKEDA |
| | | MKTKTHYHAM HADCLQELRR YLKSGVVLRR TVPPMVNVTR SEASEGNITV |
| | | TCRASGFYPW NITLSWRQDG VSLSHDTQQW GDVLPDGNGT YQTWVATRIC |
| | | QGEEQRFTCY MEHSGNHSTH PVPS |
| MICA*008 | 4 | EPHSLRYNLT VLSWDGSVQS GFLAEVHLDG QPFLRYDRQK CRAKPQGQWA |
| | | EDVLGNKTWD RETRDLTGNG KDLRMTLAHI KDQKEGLHSL QEIRVCEIHE |
| | | DNSTRSSQHF YYDGELFLSQ NLETEEWTVP QSSRAQTLAM NVRNFLKEDA |
| | | MKTKTHYHAM HADCLQELRR YLESGVVLRR TVPPMVNVTR SEASEGNITV |
| | | TCRASSFYPR NIILTWRQDG VSLSHDTQQW GDVLPDGNGT YQTWVATRIC |
| | | RGEEQRFTCY MEHSGNHSTH PVPS |
| MICA*019 | 5 | EPHSLRYNLT VLSWDGSVQS GFLAEVHLDG QPFLRYDRQK CRAKPQGQWA |
| | | EDVLGNKTWD RETRDLTGNG KDLRMTLAHI KDQKEGLHSL QEIRVCEIHE |
| | | DNSTRSSQHF YYDGELFLSQ NLETEEWTVP QSSRAQTLAM NVRNFLKEDA |
| | | MKTKTHYHAM HADCLQELRR YLESSVVLRR TVPPMVNVTR SEASEGNITV |
| | | TCRASSFYPR NIILTWRQDG VSLSHDTQQW GDVLPDGNGT YQTWVATRIC |
| | | RGEEQRFTCY MEHSGNHSTH PVPS |
| MICB | 36 | MGLGRVLLFL AVAFPFAPPA AAAEPHSLRY NLMVLSQDGS VQSGFLAEGH |
| | | LDGQPFLRYD RQKRRAKPQG QWAEDVLGAK TWDTETEDLT ENGQDLRRTL |
| | | THIKDQKGGL HSLQEIRVCE IHEDSSTRGS RHFYYDGELF LSQNLETQES |
| | | TVPQSSRAQT LAMNVTNFWK EDAMKTKTHY RAMQADCLQK LQRYLKSGVA |
| | | IRRTVPPMVN VTCSEVSEGN ITVTCRASSF YPRNITLTWR QDGVSLSHNT |
| | | QQWGDVLPDG NGTYQTWVAT RIRQGEEQRF TCYMEHSGNH GTHPVPSGKA |
| | | LVLQSQRTDF PYVSAAMPCF VIIIILCVPC CKKKTSAAEG PELVSLQVLD |
| | | QHPVGTGDHR DAAQLGFQPL MSATGSTGST EGA |

The MICA gene encodes a protein that belongs to the MhcSF and to the IgSF. This protein is a transmembrane MHC-I-alpha-like (I-alpha-like) chain, which comprises three extracellular domains, two distal G-like domains, G-alpha1-like (also referred to as “D1” or “α1”) and G-alpha2-like (also referred to as “D2” or “α2”), and a C-like-domain (also referred to as “D3” or “α3”) proximal to the cell membrane, and three regions, a connecting-region, a transmembrane-region and a cytoplasmic-region (labels according to the IMGT Scientific Chart of the IMGT (international ImMunoGeneTics information System®), http://imgt.org and LeFranc et al. In Silico Biology, 2005; 5:45-60). The MICA mature protein including leader, ECD, TM and CY domains, is made up of 360 to 366 amino acids, the difference arising from a microsatellite polymorphism in the transmembrane region. The α1, α2 and α3 can be defined according to any suitable numbering system (e.g., the IMGT numbering system). In one embodiment, the α1 domain comprises residue positions 1 to 88 of the MICA polypeptide of SEQ ID NO: 1; the α2 domain comprises residue positions 89 to 181 of the MICA polypeptide of SEQ ID NO: 1; and the α3 domain comprises residue positions 182 to 274 of the MICA polypeptide of SEQ ID NO: 1. The α1 and α2 domains each comprise A, B, C and D strands, AB, BC and CD turns, and a helix. The α3 domain comprises A, B, C, D, E, F and G strands, a BC loop, a CD strand, a DE-turn and an FG loop. The MICA protein is highly glycosylated with eight potential glycosylation sites, two in α1, one in α2 and five in the α3 domain, including 0-glycans (N-acetyllactosamine linked to serine or threonine) and/or N-glycans. While MICA is expressed constitutively in certain cells, low levels of MICA expression do not usually give rise to host immune cell attach. However, on MICA is upregulated on rapidly proliferating cells such as tumor cells. MICA is the most highly expressed of all NKG2D ligands, and it has been found across a wide range of tumor types (e.g., carcinomas in general, bladder cancer, melanoma, lung cancer, hepatocellular cancer, glioblastoma, prostate cancer, hematological malignancies in general, acute myeloid leukemia, acute lymphatic leukemia, chronic myeloid leukemia and chronic lymphatic leukemia. Recently, Tsuboi et al. (2011) (EMBO J: 1-13) reported that the 0-glycan branching enzyme, core2 β-1,6-N-acetylglucosaminyltransferase (C2GnT) is active in MICA-expressing tumor cells and that MICA from tumor cells contains core2 O-glycan (an O-glycan comprising an N-acetylglucosamine branch connected to N-acetylgalactosamine).

Bauer et al Science 285: 727-729, 1999 provided a role for MICA as a stress-inducible ligand for NKG2D. As used herein, “MICA” refers to any MICA polypeptide, including any variant, derivative, or isoform of the MICA gene or encoded protein(s) to which they refer. The MICA gene is polymorphic, displaying an unusual distribution of a number of variant amino acids in their extracellular alpha-1, alpha-2, and alpha-3 domains. Various allelic variants have been reported for MICA polypeptides (e.g., MICA), each of these are encompassed by the respective terms, including, e.g., human MICA polypeptides MICA*001, MICA*002, MICA*004, MICA*005, MICA*006, MICA*007, MICA*008, MICA*009, MICA*010, MICA*011, MICA*012, MICA*013, MICA*014, MICA*015, MICA*016, MICA*017, MICA*018, MICA*019, MICA*020, MICA*022, MICA*023, MICA*024, MICA*025, MICA*026, MICA*027, MICA*028, MICA*029, MICA*030, MICA*031, MICA*032, MICA*033, MICA*034, MICA*035, MICA*036, MICA*037, MICA*038, MICA*039, MICA*040, MICA*041, MICA*042, MICA*043, MICA*044, MICA*045, MICA*046, MICA*047, MICA*048, MICA*049, MICA*050, MICA*051, MICA*052, MICA*053, MICA*054, MICA*055, MICA*056 and further MICA alleles MICA*057-MICA*087.

As used herein, “hNKG2D” and, unless otherwise stated or contradicted by context, the terms “NKG2D,” “NKG2-D,” “CD314,” “D12S2489E,” “KLRK1,” “killer cell lectin-like receptor subfamily K, member 1,” or “KLRK1,” refer to a human killer cell activating receptor gene, its cDNA (e.g., GenBank Accession No. NM_007360), and its gene product (GenBank Accession No. NP_031386), or naturally occurring variants thereof. In NK and T cells, hNKG2D can form heterodimers or higher order complexes with proteins such as DAP10 (GenBank Accession No. AAG29425, AAD50293). Any activity attributed herein to hNKG2D, e.g., cell activation, antibody recognition, etc., can also be attributed to hNKG2D in the form of a heterodimer such as hNKG2D-DAP10, or higher order complexes with these two (and/or other) components.

The 3D structure of MICA in complex with NKG2D has been determined (see, e.g., Li et al., Nat. Immunol. 2001; 2:443-451; code 1hyr, and in IMGT/3Dstructure-DB (Kaas et al. Nucl. Acids Res. 2004; 32:D208-D210)). When MICA is in complex with a NKG2D homodimer, the residues 63 to 73 (IGMT numbering) of MICA α2 are ordered, adding almost two turns of helix. The two monomers of NKG2D equally contribute to interactions with MICA, and seven positions in each NKG2D monomer interact with one of the MICA α1 or α2 helix domains.

The invention provides methods of using the anti-MICA antibodies disclosed herein; for example, provided is a method for inhibiting cell proliferation or activity, for delivering a molecule to a cell (e.g., a toxic molecule, a detectable marker, etc.), for targeting, identifying or purifying a cell, for depleting, killing or eliminating a cell, for reducing cell proliferation, the method comprising exposing a cell, such as a tumor cell which expresses a MICA polypeptide, to an antigen-binding compound of the disclosure that binds a MICA polypeptide. It will be appreciated that for the purposes herein, “cell proliferation” can refer to any aspect of the growth or proliferation of cells, e.g., cell growth, cell division, or any aspect of the cell cycle. The cell may be in cell culture (in vitro) or in a mammal (in vivo), e.g., a mammal suffering from a MICA-expressing pathology. Also provided is a method for inducing the death of a cell or inhibiting the proliferation or activity of a cell which expresses a MICA polypeptide, comprising exposing the cell to an antigen-binding compound that binds a MICA polypeptide linked to a toxic agent, in an amount effective to induce death and/or inhibit the proliferation of the cell. Thus, also provided is a method for treating a mammal suffering from a proliferative disease, and any condition characterized by a pathogenic expansion of cells expressing of a MICA polypeptide, the method comprising administering a pharmaceutically effective amount of an antibody disclosed herein to the mammal, e.g., for the treatment of a cancer.

### Definitions

As used in the specification, “a” or “an” may mean one or more. As used in the claim(s), when used in conjunction with the word “comprising”, the words “a” or “an” may mean one or more than one. As used herein “another” may mean at least a second or more.

Where “comprising” is used, this can optionally be replaced by “consisting essentially of” or by “consisting of”.

Whenever within this whole specification “treatment of cancer” or the like is mentioned with reference to anti-MICA binding agent (e.g., antibody), there is meant: (a) method of treatment of cancer, said method comprising the step of administering (for at least one treatment) an anti-MICA binding agent, (for example in a pharmaceutically acceptable carrier material) to an individual, a mammal, especially a human, in need of such treatment, in a dose that allows for the treatment of cancer, (a therapeutically effective amount), optionally in a dose (amount) as specified herein; (b) the use of an anti-MICA binding agent for the treatment of cancer, or an anti-MICA binding agent, for use in said treatment (especially in a human); (c) the use of an anti-MICA binding agent for the manufacture of a pharmaceutical preparation for the treatment of cancer, a method of using an anti-MICA binding agent for the manufacture of a pharmaceutical preparation for the treatment of cancer, comprising admixing an anti-MICA binding agent with a pharmaceutically acceptable carrier, or a pharmaceutical preparation comprising an effective dose of an anti-MICA binding agent that is appropriate for the treatment of cancer; or (d) any combination of a), b), and c), in accordance with the subject matter allowable for patenting in a country where this application is filed.

The term “antibody,” as used herein, refers to polyclonal and monoclonal antibodies. Depending on the type of constant domain in the heavy chains, antibodies are assigned to one of five major classes: IgA, IgD, IgE, IgG, and IgM. Several of these are further divided into subclasses or isotypes, such as IgG1, IgG2, IgG3, IgG4, and the like. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids that is primarily responsible for antigen recognition. The terms variable light chain (V_{L}) and variable heavy chain (V_{H}) refer to these light and heavy chains respectively. The heavy-chain constant domains that correspond to the different classes of immunoglobulins are termed “alpha,” “delta,” “epsilon,” “gamma” and “mu,” respectively. The subunit structures and three-dimensional configurations of different classes of immunoglobulins are well known. IgG are the exemplary classes of antibodies employed herein because they are the most common antibodies in the physiological situation and because they are most easily made in a laboratory setting. Optionally the antibody is a monoclonal antibody. Particular examples of antibodies are humanized, chimeric, human, or otherwise-human-suitable antibodies. “Antibodies” also includes any fragment or derivative of any of the herein described antibodies.

The term “specifically binds to” means that an antibody can bind preferably in a competitive binding assay to the binding partner, e.g., MICA and MICB, as assessed using either recombinant forms of the proteins, epitopes therein, or native proteins present on the surface of isolated target cells. Competitive binding assays and other methods for determining specific binding are further described below and are well known in the art.

When an antibody is said to “compete with” a particular monoclonal antibody, it means that the antibody competes with the monoclonal antibody in a binding assay using either recombinant MICA molecules or surface expressed MICA molecules. For example, if a test antibody reduces the binding of a reference antibody to a MICA polypeptide or MICA-expressing cell in a binding assay, the antibody is said to “compete” respectively with the reference antibody.

The term “affinity”, as used herein, means the strength of the binding of an antibody to an epitope. The affinity of an antibody is given by the dissociation constant Kd, defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant Kₐ is defined by 1/Kd. Methods for determining the affinity of mAbs can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One standard method well known in the art for determining the affinity of mAbs is the use of surface plasmon resonance (SPR) screening (such as by analysis with a BIAcore™ SPR analytical device).

Within the context herein a “determinant” designates a site of interaction or binding on a polypeptide.

The term “epitope” refers to an antigenic determinant, and is the area or region on an antigen to which an antibody binds. A protein epitope may comprise amino acid residues directly involved in the binding as well as amino acid residues which are effectively blocked by the specific antigen binding antibody or peptide, i.e., amino acid residues within the “footprint” of the antibody. It is the simplest form or smallest structural area on a complex antigen molecule that can combine with e.g., an antibody or a receptor. Epitopes can be linear or conformational/structural. The term “linear epitope” is defined as an epitope composed of amino acid residues that are contiguous on the linear sequence of amino acids (primary structure). The term “conformational or structural epitope” is defined as an epitope composed of amino acid residues that are not all contiguous and thus represent separated parts of the linear sequence of amino acids that are brought into proximity to one another by folding of the molecule (secondary, tertiary and/or quaternary structures). A conformational epitope is dependent on the 3-dimensional structure. The term ‘conformational’ is therefore often used interchangeably with ‘structural’.

The term “deplete” or “depleting”, with respect to MICA-expressing cells, means a process, method, or compound that results in killing, elimination, lysis or induction of such killing, elimination or lysis, so as to negatively affect the number of such MICA-expressing cells present in a sample or in a subject.

The term “antibody-dependent cell-mediated cytotoxicity” or “ADCC” is a term well understood in the art, and refers to a cell-mediated reaction in which non-specific cytotoxic cells that express Fc receptors (FcRs) recognize bound antibody on a target cell and subsequently cause lysis of the target cell. Non-specific cytotoxic cells that mediate ADCC include natural killer (NK) cells, macrophages, monocytes, neutrophils, and eosinophils.

The term “complement-dependent cytotoxicity” or “CDC” is a term well understood in the art, and refers to the ability of a molecule to lyse a target in the presence of complement. The complement activation pathway is initiated by the binding of the first component of the complement system (C1q) to a molecule (e.g., an antibody) complexed with a cognate antigen.

The term “agent” is used herein to denote a chemical compound, a mixture of chemical compounds, a biological macromolecule, or an extract made from biological materials. The term “therapeutic agent” refers to an agent that has biological activity.

For the purposes herein, a “humanized” or “human” antibody refers to an antibody in which the constant and variable framework region of one or more human immunoglobulins is fused with the binding region, e.g., the CDR, of an animal immunoglobulin. Such antibodies are designed to maintain the binding specificity of the non-human antibody from which the binding regions are derived, but to avoid an immune reaction against the non-human antibody. Such antibodies can be obtained from transgenic mice or other animals that have been “engineered” to produce specific human antibodies in response to antigenic challenge (see, e.g., Green et al. (1994) Nature Genet 7:13; Lonberg et al. (1994) Nature 368:856; Taylor et al. (1994) Int Immun 6:579, the entire teachings of which are herein incorporated by reference). A fully human antibody also can be constructed by genetic or chromosomal transfection methods, as well as phage display technology, all of which are known in the art (see, e.g., McCafferty et al. (1990) Nature 348:552-553). Human antibodies may also be generated by in vitro activated B cells (see, e.g., U.S. Pat. Nos. 5,567,610 and 5,229,275, which are incorporated in their entirety by reference).

As used herein, the term “antigen binding domain” refers to a domain comprising a three-dimensional structure capable of immunospecifically binding to an epitope. Thus, in one embodiment, said domain can comprise a hypervariable region, optionally a VH and/or VL domain of an antibody chain, optionally at least a VH domain. In another embodiment, the binding domain may comprise at least one complementarity determining region (CDR) of an antibody chain. In another embodiment, the binding domain may comprise a polypeptide domain from a non-immunoglobulin scaffold.

The term “hypervariable region” when used herein refers to the amino acid residues of an antibody that are responsible for antigen binding. The hypervariable region generally comprises amino acid residues from a “complementarity-determining region” or “CDR” (e.g., residues 24-34 (L1), 50-56 (L2) and 89-97 (L3) in the light-chain variable domain and 31-35 (H1), 50-65 (H2) and 95-102 (H3) in the heavy-chain variable domain; disclosure (see Kabat et al. (1991) Sequences of Protein of Immunological Interest, 5th ed., United States Public Health Service, National Institute of Health, Bethesda, Md.)) and/or those residues from a “hypervariable loop” (e.g., residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol 1987; 196:901-917), or a similar system for determining essential amino acids responsible for antigen binding. Using the Kabat numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (residue 52a according to Kabat) after residue 52 of CDR H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence. Another suitable numbering system is the Abnum system. Unless otherwise specified, the Abnum amino acid numbering nomenclature for immunoglobulins is used to refer to positions in the VH and VL domains (see Abhinandan and Martin, (2008) Molecular Immunology 45: 3832-3839, the disclosure of which is incorporated by reference). Sequence numbering using the Abnum system can also be automatically generated at http://www.bioinfo.org.uk/abs/abnum. However it will be appreciated that the person of skill in the art can use an alternative numbering system and identify positions corresponding to Abnum numbering. Phrases such as “Abm position”, “Abm numbering” and “according to Abm” herein refer to this numbering system for heavy chain variable domains or light chain variable domains.

By “framework” or “FR” residues as used herein is meant the region of an antibody variable domain exclusive of those regions defined as CDRs. Each antibody variable domain framework can be further subdivided into the contiguous regions separated by the CDRs (FR1, FR2, FR3 and FR4).

The terms “Fc domain,” “Fc portion,” and “Fc region” refer to a C-terminal fragment of an antibody heavy chain, e.g., from about amino acid (aa) 230 to about aa 450 (Kabat numbering) of human γ (gamma) heavy chain or its counterpart sequence in other types of antibody heavy chains (e.g., α, δ, ε and μ for human antibodies), or a naturally occurring allotype thereof.

The terms “isolated”, “purified” or “biologically pure” refer to material that is substantially or essentially free from components which normally accompany it as found in its native state. Purity and homogeneity are typically determined using analytical chemistry techniques such as polyacrylamide gel electrophoresis or high performance liquid chromatography. A protein that is the predominant species present in a preparation is substantially purified.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (nonrecombinant) form of the cell or express native genes that are otherwise abnormally expressed, under expressed or not expressed at all.

Within the context herein, the term antibody that “binds” a polypeptide or epitope designates an antibody that binds said determinant with specificity and/or affinity.

The term “identity” or “identical”, when used in a relationship between the sequences of two or more polypeptides, refers to the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

Methods for determining identity are designed to give the largest match between the sequences tested. Methods of determining identity are described in publicly available computer programs. Computer program methods for determining identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well-known Smith Waterman algorithm may also be used to determine identity.

### Production of Antibodies

The present invention is based, in part, on the discovery of modified human acceptor framework sequences into which antibody CDRs can be incorporated such that the resulting anti-MICA variable region has high physicochemical stability and high binding affinity for the predominant human MICA alleles. Furthermore, provided are antibodies with high content of human amino acid sequences, thereby providing decreased risk of immunogenicity when administered to a human individual. Advantageously, the antibodies have low potential to elicit human anti-mouse antibodies (HAMA).

Anti-MICA antibody VH and VL sequences are provided below in Table 1, amino acid differing between respective VH domains and VL domains are underlined:

TABLE 1

| Antibody | |
|---|---|
| domain | Amino acid sequence |
| mAb1 | QVQLQESGPGLVKPSETLSLTCTVSGYSITSDYAWNWIRQ |
| VH | PPGKGLEWIGFVSYSGTTKYNPSLKSRVTISRDTSKNQFS |
| | LKLSSVTAADTAVYYCARGYGFDYWGQGTTVTVSS |
| | (SEQ ID NO: 6) |
| mAb1 | EIVLTQSPATLSLSPGERATLSCSATSSISSIYFHWYQQK |
| VL | PGQAPRLLIYRTSNLASGIPARFSGSGSGTDYTLTISSLE |
| | PEDFAVYYCQQGTTIPFTFGQGTKLEIK |
| | (SEQ ID NO: 7) |
| mAb2 | QVQLQESGPGLVKPSETLSLTCTVSGYSITSDYAWNWIRQ |
| VH | PPGKGLEWIGFVSYSGTTKYNPSLKSRVTISRDTSKNQFS |
| | LKLSSVTAADTAVYYCARGYGFDYWGQGTTVTVSS |
| | (SEQ ID NO: 8) |
| mAb2 | EIVLTQSPATLSLSPGERATLSCSATSSISSIYFHWYQQK |
| VL | PGQAPRLLIYRTSNLASGIPARFSGSGSGTSYTLTISSLE |
| | PEDFAVYYCQQGTTIPFTFGQGTKLEIK |
| | (SEQ ID NO: 9) |
| mAb3 | QVQLQESGPGLVKPSETLSLTCTVSGYSITSDYAWNWIRQ |
| VH | PPGKGLEWIGFVSYSGTTKYNPSLKSRVTISRDTSKNQFS |
| | LKLSSVTAADTAVYYCARGYGFDYWGQGTTVTVSS |
| | (SEQ ID NO: 10) |
| mAb3 | EIVLTQSPATLSLSPGERATLSCSATSSISSIYFHWYQQK |
| VL | PGQAPRLLIYRTSNLASGIPARFSGSGSGTDYTLTISSLE |
| | PEDVAVYYCQQGTTIPFTFGQGTKLEIK |
| | (SEQ ID NO: 11) |

Positions in the VH and VL domains herein are described using the Abnum amino acid numbering nomenclature for immunoglobulins (see Abhinandan and Martin, (2008) Molecular Immunology 45: 3832-3839, the disclosure of which is incorporated by reference). Sequence numbering using the Abnum system can also be automatically generated at http://www.bioinfo.org.uk/abs/abnum. However it will be appreciated that the person of skill in the art can use an alternative numbering system and identify positions corresponding to Abnum numbering.

In one embodiment, the antibody comprises a heavy chain framework from the human subgroup IGHV4-b (e.g., IGHV4-b*02) and the J-segment is from IGHJ6 (e.g., IGHJ6*01). In one embodiment, the humanized antibody comprises a light chain framework from the human subgroup IGKV3-11 (e.g., IGKV3-11*01) and the J-segment is from IGKJ2 (e.g., IGKJ2*01).

The antibody may further comprise one or more mutations in the human framework sequences, to, e.g., enhance affinity, stability, or other properties of the antibody.

Examples of VH and VL amino acid sequences of an anti-MICA antibody are shown in SEQ ID NOS: 6-21, respectively. In one aspect, provided is an isolated antibody that binds a human MICA polypeptide, wherein the antibody comprises: a HCDR1 region comprising an amino acid sequence SDYAWN as set forth in SEQ ID NO: 30, or a sequence of at least 3 or 4 amino acids thereof; a HCDR2 region comprising an amino acid sequence FVSYSGTTKYNPSLKS as set forth in SEQ ID NO: 31, or a sequence of at least 4, 5, 6, 7, 8, 9 or 10 contiguous amino acids thereof; a HCDR3 region comprising an amino acid sequence GYGFDY as set forth in SEQ ID NO: 32, or a sequence of at least 4, 5, 6, 7, 8, 9 or 10 contiguous amino acids thereof; a LCDR1 region comprising an amino acid sequence SATSSISSIYFH as set forth in SEQ ID NO: 33, or a sequence of at least 4, 5, 6, 7, 8, 9 or 10 contiguous amino acids thereof; a LCDR2 region comprising an amino acid sequence RTSNLA as set forth in SEQ ID NO: 34, or a sequence of at least 3, 4 or 5 contiguous amino acids thereof; a LCDR3 region comprising an amino acid sequence QQGTTIPFT as set forth in SEQ ID NO: 35, or a sequence of at least 5, 6, 7, or 8 contiguous amino acids thereof.

In one aspect, provided is an antigen binding domain or antibody that binds a human MICA polypeptide, comprising:

(a) a CDR-H1 comprising the amino acid sequence of SEQ ID NO: 30;
(b) a CDR-H2 comprising the amino acid sequence of SEQ ID NO: 31;
(c) a CDR-H3 comprising the amino acid sequence of SEQ ID NO: 32;
(d) a CDR-L1 comprising the amino acid sequence of SEQ ID NO: 33;
(e) a CDR-L2 comprising the amino acid sequence of SEQ ID NO: 34;
(f) a CDR-L3 comprising the amino acid sequence of SEQ ID NO: 35; and
(g) human heavy and light chain framework sequences,

wherein the antigen binding domain or antibody comprises a VH comprising an amino acid sequence at least 80%, 90%, 95% or 98% identical to the amino acid sequence of SEQ ID NO: 6 and a VL comprising an amino acid sequence at least 80%, 90%, 95% or 98% identical to the amino acid sequence of SEQ ID NO: 7.

In one embodiment, the light chain variable region (VL) comprises an amino acid residue at Abnum position 71 (in FR3) capable of forming a non-covalent bonds with amino acids within the CDR1 of the VL. In one embodiment, the VL comprises a tyrosine (Y) amino acid residue at Abnum position 71 (in FR3). In one embodiment, the VL comprises a phenylalanine (F) at Abnum position 83.

In one embodiment, the heavy chain variable region (VH) comprises amino acid residues at Abnum positions 72c (in FR2) and 74 (in FR3) capable of interacting with one another to form a salt bridge, e.g., H-bonding between the residue at Abnum position 72c and the residue at position 74. In one embodiment, the VH comprises a lysine (K) amino acid residue at Abnum position 72c and a glutamine residue at position 74. In one embodiment, the VH comprises a threonine (T) at Abnum position 30. In one embodiment, the VH comprises an isoleucine (I) at Abnum position 48. In one embodiment the VH comprises a valine (V) at Abnum position 67. In one embodiment, the VH comprises an arginine (R) at Abnum position 71.

In one embodiment, the VH comprises a heavy chain framework from the human subgroup IGHV4-b (e.g., IGHV4-b*02) and the J-segment is from IGHJ6 (e.g., IGHJ6*01). In one embodiment, the VL comprises a light chain framework from the human subgroup IGKV3-11 (e.g., IGKV3-11*01) and the J-segment is from IGKJ2 (e.g., IGKJ2*01).

Optionally a human VH and/or VL framework (e.g., or a heavy or light chain FR1, FR2, FR3 and/or FR4 thereof) may or may not comprises one or more mutations, e.g., back mutations to introduce a residue present at the particular position in a non-human mammal (e.g., a mouse or a rat). The antibody may or may not further comprise one or more additional mutations (e.g., back-mutations) in the human framework sequences, to, e.g., enhance affinity, stability, or other properties of the antibody.

In another aspect, provided is anti-MICA antibodies that comprise a VH domain having at least about 80% sequence identity (e.g., at least about 85%, 90%, 95%, 97%, 98%, or more identity) to the VH domain of SEQ ID NOS: 6 or 8. In another aspect, provided are anti-MICA antibodies that comprise a VL domain having at least about 80% sequence identity (e.g., at least about 85%, 90%, 95%, 97%, 98%, or more identity) to the VH domain of SEQ ID NOS: 7 or 9.

DNA encoding an antibody can be prepared and placed in an appropriate expression vector for transfection into an appropriate host. The host is then used for the recombinant production of the antibody, or variants thereof, such as a humanized version of that monoclonal antibody, active fragments of the antibody, chimeric antibodies comprising the antigen recognition portion of the antibody, or versions comprising a detectable moiety.

DNA encoding the monoclonal antibodies of the disclosure can be readily isolated and sequenced using conventional procedures (e.g., by using oligonucleotide probes that are capable of binding specifically to genes encoding the heavy and light chains of murine antibodies). In one aspect, provided is a nucleic acid encoding a heavy chain or a light chain of an anti-MICA antibody of any embodiment herein. Once isolated, the DNA can be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. As described elsewhere in the present specification, such DNA sequences can be modified for any of a large number of purposes, e.g., for humanizing antibodies, producing fragments or derivatives, or for modifying the sequence of the antibody, e.g., in the antigen binding site in order to optimize the binding specificity of the antibody. In one embodiment, provided is an isolated nucleic acid sequence encoding a light chain and/or a heavy chain of an antibody, as well as a recombinant host cell comprising (e.g., in its genome) such nucleic acid. Recombinant expression in bacteria of DNA encoding the antibody is well known in the art (see, for example, Skerra et al., Curr. Opinion in Immunol., 5, pp. 256 (1993); and Pluckthun, Immunol. 130, p. 151 (1992).

Typically, an anti-MICA antibody provided herein has an affinity for a MICA polypeptide in the range of about 10⁴ to about 10¹¹ M⁻¹ (e.g., about 10⁸ to about 10¹⁰ M⁻¹). For example, in a particular aspect the disclosure provides Anti-MICA antibody that have an average disassociation constant (K_{D}) of less than 1×10⁻⁹ M with respect to MICA, as determined by, e.g., surface plasmon resonance (SPR) screening (such as by analysis with a BIAcore™ SPR analytical device). In a more particular exemplary aspect, the disclosure provides anti-MICA antibodies that have a KD of about 1×10⁻⁸ M to about 1×10⁻¹⁰ M, or about 1×10⁻⁹ M to about 1×10¹¹ M, for MICA (e.g., MICA*001, *004, *007 and *008 alleles).

Antibodies can be characterized for example by a mean KD of no more than about (i.e. better affinity than) 100, 60, 10, 5, or 1 nanomolar, preferably sub-nanomolar or optionally no more than about 500, 200, 100 or 10 picomolar. KD can be determined for example for example by immobilizing recombinantly produced human MICA proteins on a chip surface, followed by application of the antibody to be tested in solution. In one embodiment, the method further comprises a step (d), selecting antibodies from (b) that are capable of competing for binding to MICA with antibody of the disclosure.

Where the test antibodies have modifications in their VH and/VL, a simple competition assay may be employed in which the control (the antibody having a VH and VL of SEQ ID NOS: 6 and 7, or the antibody having a VH and VL of SEQ ID NOS: 8 and 9, for example) and test antibodies are admixed (or pre-adsorbed) and applied to a sample containing MICA polypeptides. Protocols based upon western blotting and the use of Biacore™ analysis are suitable for use in such competition studies.

In certain embodiments, one pre-mixes the control antibodies with varying amounts of the test antibodies (e.g., about 1:10 or about 1:100) for a period of time prior to applying to the MICA antigen sample. In other embodiments, the control and varying amounts of test antibodies can simply be admixed during exposure to the MICA antigen sample. As long as one can distinguish bound from free antibodies (e.g., by using separation or washing techniques to eliminate unbound antibodies) and control antibody from the test antibodies (e.g., by using species-specific or isotype-specific secondary antibodies or by specifically labelling control antibody with a detectable label) one can determine if the test antibodies reduce the binding of control antibody to the antigens, indicating that the test antibody recognizes substantially the same epitope as control antibody. The binding of the (labelled) control antibodies in the absence of a completely irrelevant antibody can serve as the control high value. The control low value can be obtained by incubating the labelled control antibodies with unlabelled antibodies of exactly the same type, where competition would occur and reduce binding of the labelled antibodies. In a test assay, a significant reduction in labelled antibody reactivity in the presence of a test antibody is indicative of a test antibody that recognizes substantially the same epitope, i.e., one that “cross-reacts” or competes with the labelled control antibody. Any test antibody that reduces the binding of control antibody to MICA antigens by at least about 50%, such as at least about 60%, or more preferably at least about 80% or 90% (e.g., about 65-100%), at any ratio of control antibody:test antibody between about 1:10 and about 1:100 is considered to be an antibody that binds to substantially the same epitope or determinant as control antibody. In one embodiment, such test antibody will reduce the binding of control antibody to the MICA antigen by at least about 90% (e.g., about 95%).

Competition can also be assessed by, for example, a flow cytometry test. In such a test, cells bearing a given MICA polypeptide can be incubated first with control antibody, for example, and then with the test antibody labelled with a fluorochrome or biotin. The antibody is said to compete with control antibody if the binding obtained upon preincubation with a saturating amount of control antibody is about 80%, optionally about 50%, about 40% or less (e.g., about 30%, 20% or 10%) of the binding (as measured by mean of fluorescence) obtained by the antibody without preincubation with control antibody. Alternatively, an antibody is said to compete with control antibody if the binding obtained with a labelled control antibody antibody (by a fluorochrome or biotin) on cells preincubated with a saturating amount of test antibody is about 80%, optionally about 50%, about 40%, or less (e.g., about 30%, 20% or 10%) of the binding obtained without preincubation with the test antibody.

A simple competition assay in which a test antibody is pre-adsorbed and applied at saturating concentration to a surface onto which a MICA antigen is immobilized may also be employed. The surface in the simple competition assay is preferably a Biacore™ chip (or other media suitable for surface plasmon resonance analysis). The control antibody (the antibody having a VH and VL of SEQ ID NOS: 6 and 7, or the antibody having a VH and VL of SEQ ID NOS: 8 and 9, for example) is then brought into contact with the surface at a MICA-saturating concentration and the MICA and surface binding of the control antibody is measured. This binding of the control antibody is compared with the binding of the control antibody to the MICA-containing surface in the absence of test antibody. In a test assay, a significant reduction in binding of the MICA-containing surface by the control antibody in the presence of a test antibody indicates that the test antibody recognizes substantially the same epitope as the control antibody such that the test antibody “cross-reacts” with the control antibody. Any test antibody that reduces the binding of control antibody to a MICA antigen by at least about 30% or more, preferably about 40%, can be considered to be an antibody that binds to substantially the same epitope or determinant as control antibody. Preferably, such a test antibody will reduce the binding of the control antibody to the MICA antigen by at least about 50% (e.g., at least about 60%, at least about 70%, or more). It will be appreciated that the order of control and test antibodies can be reversed: that is, the control antibody can be first bound to the surface and the test antibody is brought into contact with the surface thereafter in a competition assay. Preferably, the antibody having higher affinity for the MICA antigen is bound to the surface first, as it will be expected that the decrease in binding seen for the second antibody (assuming the antibodies are cross-reacting) will be of greater magnitude. Further examples of such assays are provided in, e.g., Saunal (1995) J. Immunol. Methods 183: 33-41, the disclosure of which is incorporated herein by reference.

Determination of whether an antibody binds within an epitope region can be carried out in ways known to the person skilled in the art. As one example of such mapping/characterization methods, an epitope region for an anti-MICA antibody may be determined by epitope “foot-printing” using chemical modification of the exposed amines/carboxyls in the MICA protein. One specific example of such a foot-printing technique is the use of HXMS (hydrogen-deuterium exchange detected by mass spectrometry) wherein a hydrogen/deuterium exchange of receptor and ligand protein amide protons, binding, and back exchange occurs, wherein the backbone amide groups participating in protein binding are protected from back exchange and therefore will remain deuterated. Relevant regions can be identified at this point by peptic proteolysis, fast microbore high-performance liquid chromatography separation, and/or electrospray ionization mass spectrometry. See, e.g., Ehring H, Analytical Biochemistry, Vol. 267 (2) pp. 252-259 (1999) Engen, J. R. and Smith, D. L. (2001) Anal. Chem. 73, 256A-265A. Another example of a suitable epitope identification technique is nuclear magnetic resonance epitope mapping (NMR), where typically the position of the signals in two-dimensional NMR spectra of the free antigen and the antigen complexed with the antigen binding peptide, such as an antibody, are compared. The antigen typically is selectively isotopically labeled with 15N so that only signals corresponding to the antigen and no signals from the antigen binding peptide are seen in the NMR-spectrum. Antigen signals originating from amino acids involved in the interaction with the antigen binding peptide typically will shift position in the spectrum of the complex compared to the spectrum of the free antigen, and the amino acids involved in the binding can be identified that way. See, e.g., Ernst Schering Res Found Workshop. 2004; (44): 149-67; Huang et al., Journal of Molecular Biology, Vol. 281 (1) pp. 61-67 (1998); and Saito and Patterson, Methods. 1996 June; 9 (3): 516-24.

Epitope mapping/characterization also can be performed using mass spectrometry methods. See, e.g., Downard, J Mass Spectrom. 2000 April; 35 (4): 493-503 and Kiselar and Downard, Anal Chem. 1999 May 1; 71 (9): 1792-1801. Protease digestion techniques also can be useful in the context of epitope mapping and identification. Antigenic determinant-relevant regions/sequences can be determined by protease digestion, e.g., by using trypsin in a ratio of about 1:50 to MICA or o/n digestion at and pH 7-8, followed by mass spectrometry (MS) analysis for peptide identification. The peptides protected from trypsin cleavage by the anti-MICA binder can subsequently be identified by comparison of samples subjected to trypsin digestion and samples incubated with antibody and then subjected to digestion by e.g., trypsin (thereby revealing a footprint for the binder). Other enzymes like chymotrypsin, pepsin, etc., also or alternatively can be used in similar epitope characterization methods. Moreover, enzymatic digestion can provide a quick method for analyzing whether a potential antigenic determinant sequence is within a region of the MICA polypeptide that is not surface exposed and, accordingly, most likely not relevant in terms of immunogenicity/antigenicity.

Site-directed mutagenesis is another technique useful for elucidation of a binding epitope. For example, in “alanine-scanning”, each residue within a protein segment is replaced with an alanine residue, and the consequences for binding affinity measured. If the mutation leads to a significant reduction in binding affinity, it is most likely involved in binding. Monoclonal antibodies specific for structural epitopes (i.e., antibodies which do not bind the unfolded protein) can be used to verify that the alanine-replacement does not influence over-all fold of the protein. See, e.g., Clackson and Wells, Science 1995; 267:383-386; and Wells, Proc Natl Acad Sci USA 1996; 93:1-6.

Electron microscopy can also be used for epitope “foot-printing”. For example, Wang et al., Nature 1992; 355:275-278 used coordinated application of cryoelectron microscopy, three-dimensional image reconstruction, and X-ray crystallography to determine the physical footprint of a Fab-fragment on the capsid surface of native cowpea mosaic virus.

Other forms of “label-free” assay for epitope evaluation include surface plasmon resonance (SPR, Biacore™) and reflectometric interference spectroscopy (RifS). See, e.g., Fagerstam et al., Journal Of Molecular Recognition 1990; 3:208-14; Nice et al., J. Chroma-togr. 1993; 646:159-168; Leipert et al., Angew. Chem. Int. Ed. 1998; 37:3308-3311; Kroger et al., Biosensors and Bioelectronics 2002; 17:937-944.

It should also be noted that an antibody binding the same or substantially the same epitope as an antibody can be identified in one or more of the exemplary competition assays described herein. In one embodiment, a blocking α1α2 domain antibody binds an epitope comprising one, two or three residues selected from the group consisting of E100, D101 and N102, one, two or three residues selected from the group consisting of S103, T104 and R105, one or two residues selected from the group consisting of N121 and E123, and/or one or two residues selected from the group consisting of T124 and E126. In one embodiment, a blocking α1α2 domain antibody binds an epitope on a human MICA polypeptide comprising 1, 2, 3, 4, 5, 6, or more residues selected from the group consisting of residues (with reference to SEQ ID NO: 1): E100, D101, N102, S103, T104, R105, N121, E123, T124 and E126.

In one embodiment, the anti-MICA antibody has decreased binding to a mutant human MICA polypeptide having E100A, D101S, N102A substitutions (compared to a wild-type human MICA polypeptide of SEQ ID NO: 1. In one embodiment, the anti-MICA antibody has decreased binding to a mutant human MICA polypeptide having S103A, T104S, R105A substitutions (compared to a wild-type human MICA polypeptide of SEQ ID NO: 1. In one embodiment, the anti-MICA antibody has decreased binding to a mutant human MICA polypeptide having N121A, E123S, substitutions (compared to a wild-type human MICA polypeptide of SEQ ID NO: 1. In one embodiment, the anti-MICA antibody has decreased binding to a mutant human MICA polypeptide having T124A and E126A substitutions (compared to a wild-type human MICA polypeptide of SEQ ID NO: 1.

In one embodiment, the anti-MICA antibody binds to a MICA polypeptide at least partly within the α2 domain of MICA. Optionally, the antibody binds to the α2 domain at the lateral side of MICA near the NKG2D binding surface, consistent with the finding that the antibody block the interaction of cell surface MICA with NKG2D.

In view of the ability of the anti-MICA antibodies to induce ADCC and CDC, the antibodies can advantageously be made with modifications that increase their ability to bind Fc receptors which can affect effector functions such as antibody-dependent cytotoxicity, mast cell degranulation, and phagocytosis, as well as immunomodulatory signals such as regulation of lymphocyte proliferation and antibody secretion. Typical modifications include modified human IgG1 constant regions comprising at least one amino acid modification (e.g., substitution, deletions, insertions), and/or altered types of glycosylation, e.g., hypofucosylation. Such modifications can affect interaction with Fc receptors: FcγRI (CD64), FcγRII (CD32), and FcγRIII (CD 16). FcγRI (CD64), FcγRIIA (CD32A) and FcγRIII (CD 16) are activating (i.e., immune system enhancing) receptors while FcγRIIB (CD32B) is an inhibiting (i.e., immune system dampening) receptor. A modification may, for example, increase binding of the Fc domain to FcγRIIIa on effector (e.g., NK) cells.

Anti-MICA antibodies may comprise an Fc domain (or portion thereof) of human IgG1 or IgG3 isotype, optionally modified. The amino acid sequence of positions 230 to 447 sequence of a human IgG1 Fc region (GenBank accession #: J00228) is shown as follows:

(SEQ ID NO: 37)
PAPELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSVMH
EALHNHYTQKSLSLSPGK.

Residues 230-341 (Kabat EU) are the Fc CH2 region. Residues 342-447 (Kabat EU) are the Fc CH3 region. Anti-MICA antibodies may comprise a variant Fc region having one or more amino acid modifications (e.g., substitutions, deletions, insertions) in one or more portions, which modifications increase the affinity and avidity of the variant Fc region for an FcγR (including activating and inhibitory FcγRs). In some embodiments, said one or more amino acid modifications increase the affinity of the variant Fc region for FcγRIIIA and/or FcγRIIA. In another embodiment, the variant Fc region further specifically binds FcγRIIB with a lower affinity than does the Fc region of the comparable parent antibody (i.e., an antibody having the same amino acid sequence as the antibody herein except for the one or more amino acid modifications in the Fc region). For example, the one or both of the histidine residues at Kabat amino acid positions 310 and 435 may be substituted, for example by lysine, alanine, glycine, valine, leucine, isoleucine, proline, methionine, tryptophan, phenylalanine, serine or threonine (see, e.g., PCT publication no. WO 2007/080277); such substituted constant regions provide decreased binding to the inhibitory FcγRIIB without decreasing binding to the activatory FcγRIIIA. In some embodiments, such modifications increase the affinity of the variant Fc region for FcγRIIIA and/or FcγRIIA and also enhance the affinity of the variant Fc region for FcγyRIIB relative to the parent antibody. In other embodiments, said one or more amino acid modifications increase the affinity of the variant Fc region for FcγRIIIA and/or FcγRIIA but do not alter the affinity of the variant Fc regions for FcγRIIB relative to the Fc region of the parent antibody. In another embodiment, said one or more amino acid modifications enhance the affinity of the variant Fc region for FcγRIIIA and FcγRIIA but reduce the affinity for FcγRIIB relative to the parent antibody. Increased affinity and/or avidity results in detectable binding to the FcγR or FcγR-related activity in cells that express low levels of the FcγR when binding activity of the parent molecule (without the modified Fc region) cannot be detected in the cells.

In one embodiment, said one or more modifications to the amino acids of the Fc region reduce the affinity and avidity of the antibody for one or more FcγR receptors. In a specific embodiment, antibodies comprise a variant Fc region, wherein said variant Fc region comprises at least one amino acid modification relative to a wild type Fc region, which variant Fc region only binds one FcγR, wherein said FcγR is FcγRIIIA or FcγRIIA.

Specific mutations in IgG1 which affect (enhance) FcγRIIIa or FcRn binding are also set forth below.

| | | | Effector | Effect of |
|---|---|---|---|---|
| Isotype | Species | Modification | Function | Modification |
| IgG1 | Human | T250Q/M428L | Increased | Increased |
| | | | binding to FcRn | half-life |
| IgG1 | Human | 1M252Y/S254T/ | Increased | Increased |
| | | T256E + H433K/ | binding to FcRn | half-life |
| | | N434F | | |
| IgG1 | Human | E333A | Increased | Increased |
| | | | binding to | ADCC and |
| | | | FcγRIIIa | CDC |
| IgG1 | Human | S239D/A330L/ | Increased | Increased |
| | | I332E | binding to | ADCC |
| | | | FcγRIIIa | |
| IgG1 | Human | P257I/Q311 | Increased | Unchanged |
| | | | binding to FcRn | half-life |
| IgG1 | Human | S239D/I332E/ | Increased | Increased |
| | | G236A | FcγRIIa/ | macrophage |
| | | | FcγRIIb | phagocytosis |
| | | | ratio | |

The affinities and binding properties of the antibodies for an FcγR can be determined using in vitro assays (biochemical or immunological based assays) known in the art for determining antibody-antigen or Fc-FcγR interactions, i.e., specific binding of an antigen to an antibody or specific binding of an Fc region to an FcγR, respectively, including but not limited to ELISA assay, surface plasmon resonance assay, immunoprecipitation assays.

In some embodiments, the antibodies comprising a variant Fc region comprise at least one amino acid modification (for example, possessing 1, 2, 3, 4, 5, 6, 7, 8, 9, or more amino acid modifications) in the CH3 domain of the Fc region. In other embodiments, the antibodies comprise a variant Fc region comprising at least one amino acid modification (for example, possessing 1, 2, 3, 4, 5, 6, 7, 8, 9, or more amino acid modifications) in the CH2 domain of the Fc region. In some embodiments, the antibodies comprise at least two amino acid modifications (for example, possessing 2, 3, 4, 5, 6, 7, 8, 9, or more amino acid modifications), wherein at least one such modification is in the CH3 region and at least one such modification is in the CH2 region. Optionally, an antibody may comprise an amino acid modification in the hinge region. In one embodiment, provided are amino acid modification in the CH1 domain of the Fc region, optionally within a span of amino acids from Kabat positions 216-230 (Kabat EU numbering).

Any combination of Fc modifications can be made, for example any combination of different modifications disclosed in U.S. Pat. Nos. 7,632,497; 7,521,542; 7,425,619; 7,416,727; 7,371,826; 7,355,008; 7,335,742; 7,332,581; 7,183,387; 7,122,637; 6,821,505 and 6,737,056; in PCT Publications Nos. WO2011/109400; WO 2008/105886; WO 2008/002933; WO 2007/021841; WO 2007/106707; WO 06/088494; WO 05/1 15452; WO 05/110474; WO 04/1032269; WO 00/42072; WO 06/088494; WO 07/024249; WO 05/047327; WO 04/099249 and WO 04/063351; and in Presta, L. G. et al. (2002) Biochem. Soc. Trans. 30(4):487-490; Shields, R. L. et al. (2002) J. Biol. Chem. 26; 277(30):26733-26740 and Shields, R. L. et al. (2001) J. Biol. Chem. 276(9):6591-6604).

The disclosure provides anti-MICA antibodies a which comprise a variant Fc region, wherein the variant Fc region comprises at least one amino acid modification (for example, possessing 1, 2, 3, 4, 5, 6, 7, 8, 9, or more amino acid modifications) relative to a wild-type Fc region, such that the molecule has an enhanced effector function relative to a molecule comprising a wild-type Fc region, optionally wherein the variant Fc region comprises a substitution at any one or more of positions 221, 243, 247, 255, 256, 258, 267, 268, 269, 270, 272, 276, 278, 280, 283, 285, 286, 289, 290, 292, 293, 294, 295, 296, 298, 300, 301, 303, 305, 307, 308, 309, 310, 311, 312, 316, 320, 322, 326, 329, 330, 332, 331, 333, 334, 335, 337, 338, 339, 340, 359, 360, 370, 373, 376, 378, 392, 396, 399, 402, 404, 416, 419, 421, 430, 434, 435, 437, 438 and/or 439.

The disclosure provides anti-MICA antibodies a which comprise a variant Fc region, wherein the variant Fc region comprises at least one amino acid modification (for example, possessing 1, 2, 3, 4, 5, 6, 7, 8, 9, or more amino acid substitutions) relative to a wild-type Fc region, such that the molecule has an enhanced effector function relative to a molecule comprising a wild-type Fc region, optionally wherein the variant Fc region comprises a substitution at any one or more of Kabat positions 329, 298, 330, 332, 333 and/or 334 (e.g., S239D, S298A, A330L, I332E, E333A and/or K334A substitutions). In one embodiment, antibodies having variant or wild-type Fc regions may have altered glycosylation patterns that increase Fc receptor binding ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies to thereby produce an antibody with altered glycosylation. See, for example, Shields, R. L. et al. (2002) J. Biol. Chem. 277:26733-26740; Umana et al. (1999) Nat. Biotech. 17:176-1, as well as, European Patent No: EP 1,176,195; PCT Publications WO 06/133148; WO 03/035835; WO 99/54342, each of which is incorporated herein by reference in its entirety.

Generally, such antibodies with altered glycosylation are “glyco-optimized” such that the antibody has a particular N-glycan structure that produces certain desireable properties, including but not limited to, enhanced ADCC and effector cell receptor binding activity when compared to non-modified antibodies or antibodies having a naturally occurring constant region and produced by murine myeloma NSO and Chinese Hamster Ovary (CHO) cells (Chu and Robinson, Current Opinion Biotechnol. 2001, 12: 180-7), HEK293T-expressed antibodies as produced herein in the Examples section, or other mammalian host cell lines commonly used to produce recombinant therapeutic antibodies.

Monoclonal antibodies produced in mammalian host cells contain an N-linked glycosylation site at Asn297 of each heavy chain. Glycans on antibodies are typically complex biatennary structures with very low or no bisecting N-acetylglucosamine (bisecting GlcNAc) and high levels of core fucosylation. Glycan temini contain very low or no terminal sialic acid and variable amounts of galactose. For a review of effects of glycosylation on antibody function, see, e.g., Wright & Morrison, Trend Biotechnol. 15:26-31 (1997). Considerable work shows that changes to the sugar composition of the antibody glycan structure can alter Fc effector functions. The important carbohydrate structures contributing to antibody activity are believed to be the fucose residues attached via alpha-1,6 linkage to the innermost N-acetylglucosamine (GlacNAc) residues of the Fc region N-linked oligosaccharides (Shields et al., 2002).

FcγR binding requires the presence of oligosaccharides covalently attached at the conserved Asn297 in the Fc region of human IgG1, IgG2 or IgG3 type. Non-fucosylated oligosaccharides structures have recently been associated with dramatically increased in vitro ADCC activity. “Asn 297” refers to the amino acid asparagine located at about position 297 in the Fc region; based on minor sequence variations of antibodies, Asn297 can also be located some amino acids (usually not more than +3 amino acids) upstream or downstream.

Historically, antibodies produced in CHO cells contain about 2 to 6% of species that are non-fucosylated. YB2/0 (rat myeloma) and Lecl3 cell line (a lectin mutant of CHO line which has a deficient GDP-mannose 4,6-dehydratase leading to the deficiency of GDP-fucose or GDP sugar intermediates that are the substrate of alpha6-fucosyltransferase have been reported to produce antibodies with 78 to 98% non-fucosylated species. In other examples, RNA interference (RNAi) or knock-out techniques can be employed to engineer cells to either decrease the FUT8 mRNA transcript levels or knock out gene expression entirely, and such antibodies have been reported to contain up to 70% non-fucosylated glycan.

The disclosure provides an antibody binding to MICA being glycosylated with a sugar chain at Asn297, said antibody showing increased binding affinity via its Fc portion to FcγRIII. In one embodiment, an antibody will comprise a constant region comprising at least one amino acid alteration in the Fc region that improves antibody binding to FcγRIIIa and/or ADCC.

In one aspect, the antibodies are hypofucosylated in their constant region. Such antibodies may comprise an amino acid alteration or may not comprise an amino acid alteration but be produced or treated under conditions so as to yield such hypofucosylation. In one aspect, an antibody composition comprises a chimeric, human or humanized antibody described herein, wherein at least 20, 30, 40, 50, 60, 75, 85, 90, 95% or substantially all of the antibody species in the composition have a constant region comprising a core carbohydrate structure (e.g., complex, hybrid and high mannose structures) which lacks fucose. In one embodiment, provided is an antibody composition which is free of antibodies comprising a core carbohydrate structure having fucose. The core carbohydrate will preferably be a sugar chain at Asn297.

In one embodiment, disclosed is an antibody composition, e.g., a composition comprising antibodies which bind to MICA, are glycosylated with a sugar chain at Asn297, wherein the antibodies are partially fucosylated. Partially fucosylated antibodies are characterized in that the proportion of anti-MICA antibodies in the composition that lack fucose within the sugar chain at Asn297 is between 20% and 90%, for example between 20% and 80%, for example between 20% and 50%, 55%, 60%, 70% or 75%, between 35% and 50%, 55%, 60%, 70% or 75%, or between 45% and 50%, 55%, 60%, 70% or 75%. Optionally the antibody is of human IgG1 or IgG3 type.

The sugar chain show can further show any characteristics (e.g., presence and proportion of complex, hybrid and high mannose structures), including the characteristics of N-linked glycans attached to Asn297 of an antibody from a human cell, or of an antibody recombinantly expressed in a rodent cell, murine cell (e.g., CHO cell) or in an avian cell.

In one embodiment, the antibody is expressed in a cell that is lacking in a fucosyltransferase enzyme such that the cell line produces proteins lacking fucose in their core carbohydrates. For example, the cell lines Ms704, Ms705, and Ms709 lack the fucosyltransferase gene, FUT8 (alpha (1,6) fucosyltransferase), such that antibodies expressed in the Ms704, Ms705, and Ms709 cell lines lack fucose on their core carbohydrates. These cell lines were created by the targeted disruption of the FUT8 gene in CHO/DG44 cells using two replacement vectors (see U.S. Patent Publication No. 20040110704 by Yamane et al.; and Yamane-Ohnuki et al. (2004) Biotechnol Bioeng 87:614-22, the disclosures of which are incorporated herein by reference). Other examples have included use of antisense suppression, double-stranded RNA (dsRNA) interference, hairpin RNA (hpRNA) interference or intron-containing hairpin RNA (ihpRNA) interference to functionally disrupt the FUT8 gene. In one embodiment, the antibody is expressed in a cell line with a functionally disrupted FUT8 gene, which encodes a fucosyl transferase, such that antibodies expressed in such a cell line exhibit hypofucosylation by reducing or eliminating the alpha 1,6 bond-related enzyme.

In one embodiment, the antibody is expressed in cell lines engineered to express glycoprotein-modifying glycosyl transferases (e.g., beta(1,4)-N-acetylglucosaminyltransferase III (GnTHI)) such that antibodies expressed in the engineered cell lines exhibit increased bisecting GlcNac structures which results in increased ADCC activity of the antibodies (PCT Publication WO 99/54342 by Umana et al.; and Umana et al. (1999) Nat. Biotech. 17:176-180, the disclosures of which are incorporated herein by reference).

In another embodiment, the antibody is expressed and the fucosyl residue(s) is cleaved using a fucosidase enzyme. For example, the fucosidase alpha-L-fucosidase removes fucosyl residues from antibodies (Tarentino, et al. (1975) Biochem. 14:5516-5523). In other examples, a cell line producing an antibody can be treated with a glycosylation inhibitor; Zhou et al. Biotech. and Bioengin. 99: 652-665 (2008) described treatment of CHO cells with the alpha-mannosidase 1 inhibitor, kifunensine, resulting in the production of antibodies with non-fucosylated oligomannose-type N-glucans.

In one embodiment, the antibody is expressed in a cell line which naturally has a low enzyme activity for adding fucosyl to the N-acetylglucosamine that binds to the Fc region of the antibody or does not have the enzyme activity, for example the rat myeloma cell line YB2/0 (ATCC CRL 1662). Other example of cell lines include a variant CHO cell line, Led 3 cells, with reduced ability to attach fucosyl to Asn(297)-linked carbohydrates, also resulting in hypofucosylation of antibodies expressed in that host cell (WO 03/035835 (Presta et al); and Shields, R X. et al. (2002) J. Biol. Chem. 277:26733-26740, the disclosures of which are incorporated herein by reference). In another embodiment, the antibody is expressed in an avian cell, optionally a EBx® cell (Vivalis, France) which naturally yields antibodies with low fucose content e.g., WO2008/142124. Hypofucosylated glycans can also be produced in cell lines of plant origin, e.g., WO 07/084926A2 (Biolex Inc.), WO 08/006554 (Greenovation Biotech GMBH), the disclosures of which are incorporated herein by reference.

In one embodiment, the antibody comprises an Fc domain comprising an amino acid substitution that confers decreased sensitivity to cleavage by proteases. Matrix metalloproteinases (MMPs) represent the most prominent family of proteinases associated with tumorigenesis. While cancer cells can express MMPs, the bulk of the extracellular MMP is provided by different types of stromal cells that infiltrate the tumor and each produce a specific set of proteinases and proteinase inhibitors, which are released into the extracellular space and specifically alter the milieu around the tumor. The MMPs present in the tumor microenvironment can cleave antibodies within the hinge region and may thus lead to the inactivation of therapeutic antibodies that are designed to function within the tumor site. In one embodiment, the Fc domain comprising an amino acid substitution has decreased sensitivity to cleavage by any one, two, three or more (or all of) of the proteases selected from the group consisting of: GluV8, IdeS, gelatinase A (MMP2), gelatinase B (MMP-9), matrix metalloproteinase-7 (MMP-7), stromelysin (MMP-3), and macrophage elastase (MMP-12). In one embodiment, the antibody decreased sensitivity to cleavage comprises an Fc domain comprising an amino acid substitution at residues E233-L234 and/or L235. In one embodiment, the antibody comprises an Fc domain comprising an amino acid substitution at Kabat residues E233, L234, L235 and G236. In one embodiment, the antibody comprises an Fc domain comprising an amino acid substitution at one or more residues 233-238, e.g., such that E233-L234-L235-G236 sequence is replaced by P233-V234-A235 (G236 is deleted). See, e.g., WO99/58572 and WO2012087746, the disclosures of which are incorporated herein by reference.

Once an antigen-binding compound is obtained it can be assessed for its ability to block an interaction between NKG2D and MICA (e.g., membrane bound MICA), to inhibit membrane bound MICA-induced down-modulation of NKG2D on NK or CD8 T cells, to cause the death of a MICA-expressing cell (e.g., a tumor cell), to induce ADCC or CDC towards, and/or to inhibit the proliferation of and/or cause the elimination of MICA-expressing target cells.

Assessing the antigen-binding compound's ability to reduce binding or block an interaction between MICA and NKG2D can be carried out at any suitable stage of the method, e.g., as in the examples in PCT publication no. WO2013/117647. For example, tumor cells expressing MICA on their surface can be brought into contact with cells (e.g., effector cells) expressing NKG2D on their surface, with or without the addition of a candidate anti-MICA antibody. Binding between the MICA- and NKG2D-expressing cells can be assessed, and an antibody that does not reduce binding is selected. Another possibility involves contacting an isolated MICA polypeptide with an isolated NKG2D polypeptide, or a cell expressing an NKG2D polypeptide at its surface, and assessing binding between MICA and NKG2D polypeptide or cells expressing NKG2D. Another possibility involves contacting an isolated NKG2D polypeptide with a cell expressing a MICA polypeptide at its surface, and assessing binding between MICA polypeptide or a cell expressing MICA.

For example, to determine whether an agent blocks MICA interactions with NKG2D, the following test is performed: The cell line C1R or RMA transfected with MICA is incubated with a soluble NKG2D-Fc fusion protein, in the presence or absence of increasing concentrations of a test anti-MICA mAb. The cells are washed, and then incubated with a secondary antibody that recognizes the Fc part of the NKG2D-Fc fusion protein, washed again, and analyzed on a flow cytometer (FACScalibur, Beckton Dickinson), by standard methods. In the absence of anti-MICA mAbs, the NKG2D-Fc protein binds well to C1R or RMA cells. In the presence of an anti-MICA mAb that blocks MICA binding to NKG2D, there is a reduction of binding of NKG2D-Fc to the cells.

In one embodiment, assessing the antigen-binding compound's ability to reduce binding or block an interaction between MICA and NKG2D can also be carried out by assessing the effect of the anti-MICA antibody on the function of NKG2D-expressing cells (e.g., NK or T cells). Optionally, NK or T cells are used that express NKG2D but not CD16 so as to avoid any contribution of a CD16-mediated ADCC effect. If an anti-MICA antibody reduces or blocks MICA-NKG2D interactions it will be expected to dampen NKG2D-mediated activation of NK or T cells. An antibody that does not reduce binding or block an interaction between MICA and NKG2D will therefore not substantially reduce or block NKG2D-mediated activation of NK or T cells. This can be evaluated by a typical cytotoxicity assay, examples of which are described herein. Any of a number of cell-based assays can be used to assess NKG2D activity, including gene expression-based activities, cytotoxicity-based assays, and proliferation assays. In one aspect, in vitro assays will use NK cells or T cells from human patients, or, e.g., T cell lines transfected with an NKG2D-encoding transgene, so long that the expression of the receptor alters the activity of the cells in a detectable way, e.g., renders them activatable by NKG2D ligand. Any suitable physiological change that reflects NKG2D activity can be used to assess the utility of a test compound or antibody. For example, one can measure a variety of effects, such as changes in gene expression, cytokine production, cell growth, cell proliferation, pH, intracellular second messengers, e.g., Ca2+, IP3, cGMP, or cAMP, or activity such as cytotoxic activity or ability to activate other T cells. In one embodiment, the activity of the receptor is assessed by detecting the expression of NKG2D-responsive genes, e.g., CD25, IFN-gamma, or TNF-alpha (see, e.g., Groh et al. (2003) PNAS 100: 9452-9457; André et al. (2004) Eur. J. Immunol 34: 1-11). In one embodiment, NKG2D activity is assessed by incubating NKG2D+ T or NK cells in the presence of MICA-expressing cells and an anti-MICA antibody, and assessing the ability of the compound or test antibody to inhibit the release of TNF-alpha or IFN-gamma by the T or NK cells.

Exemplary cytotoxicity assays are also described in the examples herein where NKG2D-mediated killing of target cells is assessed. Here, the ability of anti-MICA antibodies to reduce or inhibit primary NK cell-mediated killing of MICA*001 or MICA*008-transfected C1R by measuring target cell release of ⁵¹Cr. The in vitro cytotoxicity assay is carried out by standard methods that are well known in the art, as described for example in Coligan et al., eds., Current Protocols in Immunology, Greene Publishing Assoc. and Wiley Interscience, N.Y., (1992, 1993). The MICA-expressing target cells are labelled with ⁵¹Cr prior to addition of NK cells, and then the killing is estimated as proportional to the release of ⁵¹Cr from the cells to the medium, as a result of killing.

Assessing the antigen-binding compound's ability to induce ADCC, CDC or otherwise (e.g., by delivery of a toxic agent) lead to the elimination or inhibition of activity of MICA-expressing target cells, can be carried out at any suitable stage. This assessment can be useful at one or more of the various steps involved in modification, production and/or development of an antibody (or other compound) destined for therapeutic use. For example, activity may be assessed where an antigen-binding compound is modified, where a cell expressing the antigen-binding compound (e.g., a host cell expressing a recombinant antigen-binding compound) has been obtained and is assessed for its ability to produce functional antibodies (or other compounds), and/or where a quantity of antigen-binding compound has been produced and is to be assessed for activity (e.g., to test batches or lots of product). Generally the antigen-binding compound will be known to specifically bind to a MICA polypeptide. The step may involve testing a plurality (e.g., a very large number using high throughput screening methods or a smaller number) of antigen-binding compounds.

Testing CDC and ADCC can be carried out can be determined by various assays including those described in the experimental examples herein. Testing ADCC typically involves assessing cell-mediated cytotoxicity in which a MICA-expressing target cell (e.g., a cancer or other MICA-expressing cell) with bound anti-MICA antibody is recognized by an effector cell (e.g., a leukocyte bearing Fc receptors), without the involvement of complement. A cell which does not express a MICA antigen can optionally be used as a control. Activation of NK cell cytotoxicity is assessed by measuring an increase in cytokine production (e.g., IFN-γ production) or cytotoxicity markers (e.g., CD107 mobilization). Optionally the antibody will induce an increase in cytokine production, expression of cytotoxicity markers, or target cell lysis of at least 20%, 50%, 80%, 100%, 200% or 500% in the presence of target (MICA-expressing) cells, compared to a control antibody (e.g., an antibody not binding to MICA, a MICA antibody having murine constant regions). In another example, lysis of target cells is detected, e.g., in a chromium release assay, optionally the antibody will induce lysis of at least 10%, 20%, 30%, 40% or 50% of target cells.

Fragments and derivatives of antibodies (which are encompassed by the term “antibody” or “antibodies” as used in this application, unless otherwise stated or clearly contradicted by context) can be produced by techniques that are known in the art. “Fragments” comprise a portion of the intact antibody, generally the antigen binding site or variable region. Examples of antibody fragments include Fab, Fab′, Fab′-SH, F (ab′) 2, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”).

In one aspect, provided is a multispecific (e.g., bispecific) antibody or antigen binding protein comprising a hypervariable region (e.g., a VH and a VL) of an antibody of any of the embodiments herein and a hypervariable region (e.g., a VH and a VL) that binds to an antigen of interest (other than MICA). In one aspect the antigen of interest is a receptor (e.g., an activating receptor) expressed at the surface of an immune effector cell (e.g., an NK cell or a T cell). In one aspect, provided is a protein or polypeptide comprising a hypervariable region.

Also encompassed are antibodies or antibody fragments of the disclosure expressed by a cell, and methods of treatment of cancer that make use of such. For example, a cell expressing a chimeric antigen receptor (CAR) can be constructed. CARs are typically engineered to comprise an extracellular single chain antibody (scFv) fused to the intracellular signaling domain of the T cell antigen receptor complex zeta chain, and have the ability, when expressed in effector cells such as T cells or NK cells, to redirect antigen recognition based on the monoclonal antibody's specificity. In one aspect, provided are genetically engineered immune cells which express and bear on the cell surface membrane a MICA-specific chimeric immune receptor comprising an intracellular signaling domain, a transmembrane domain (TM) and a MICA-specific extracellular domain (e.g., a domain derived from or comprising an antibody or antibody fragment or a variable heavy and light chain regions of the a monoclonal antibody that binds specifically to MICA). In one embodiment, the VH and VL are a VH and VL or the present disclosure. Also provided is the MICA specific chimeric immune receptors, DNA constructs encoding the receptors, and plasmid expression vectors containing the constructs in proper orientation for expression.

An anti-MICA antibody can be incorporated in a pharmaceutical formulation comprising in a concentration from 1 mg/ml to 500 mg/ml, wherein said formulation has a pH from 2.0 to 10.0. The formulation may further comprise a buffer system, preservative(s), tonicity agent(s), chelating agent(s), stabilizers and surfactants. In one embodiment, the pharmaceutical formulation is an aqueous formulation, i.e., formulation comprising water. Such formulation is typically a solution or a suspension. In a further embodiment, the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water. In another embodiment, the pharmaceutical formulation is a freeze-dried formulation, whereto the physician or the patient adds solvents and/or diluents prior to use. In another embodiment, the pharmaceutical formulation is a dried formulation (e.g., freeze-dried or spray-dried) ready for use without any prior dissolution. In a further aspect, the pharmaceutical formulation comprises an aqueous solution of such an antibody, and a buffer, wherein the antibody is present in a concentration from 1 mg/ml or above, and wherein said formulation has a pH from about 2.0 to about 10.0. In a another embodiment, the pH of the formulation is in the range selected from the list consisting of from about 2.0 to about 10.0, about 3.0 to about 9.0, about 4.0 to about 8.5, about 5.0 to about 8.0, and about 5.5 to about 7.5. In a further embodiment, the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment. In a further embodiment, the formulation further comprises a pharmaceutically acceptable preservative. In a further embodiment, the formulation further comprises an isotonic agent. In a further embodiment, the formulation also comprises a chelating agent. In a further embodiment the formulation further comprises a stabilizer. In a further embodiment, the formulation further comprises a surfactant. For convenience reference is made to Remington: The Science and Practice of Pharmacy, 19th edition, 1995. It is possible that other ingredients may be present in the peptide pharmaceutical formulation. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical formulation.

Pharmaceutical compositions containing an antibody may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen. Administration of pharmaceutical compositions may be through several routes of administration, for example, subcutaneous, intramuscular, intraperitoneal, intravenous, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

### Diagnosis and Treatment of Malignancies

Provided in one aspect are pharmaceutical compositions that comprise an antigen-binding agent (e.g., an antibody) according to the disclosure which specifically binds to MICA polypeptides on the surface of cells. The antibody in one embodiment inhibits the growth or activity (e.g. immunosuppressive activity) of the cells and/or leads to the elimination of the MICA positive cells, optionally via induction of CDC and/or ADCC. The composition further comprises a pharmaceutically acceptable carrier.

Provided in one aspect is a method of inhibiting the growth or activity of, and/or depleting, MICA-positive cells, in a human individual in need thereof, comprising the step of administering to said individual a composition according to the disclosure. Such treatment methods can be used for a number of disorders, including, but not limited to the treatment of cancers.

Provided in one aspect is a method of eliminating or inhibiting the immunosuppressive activity of MICA-positive immune cells, optionally MDSC or M2 macrophages, optionally tumor-infiltrating immunosuppressive immune cells, in a human individual in need thereof, comprising the step of administering to said individual a composition according to the disclosure.

Provided in one aspect is a method of eliminating and/or reducing the immunosuppressive activity of MICA-positive cancer cells, in a human individual in need thereof, comprising the step of administering to said individual a composition according to the disclosure.

In one embodiment, the same administration regimen is used to treat individuals whose cells express MICA*001, individuals whose cells express MICA*004, individuals whose cells express MICA*007 and individuals whose cells express MICA*008. In one embodiment, the administration regimen comprises the same mode of administration, the same dosage and the same frequency of administration irrespective of the particular allele of MICA expressed in an individual (or an individual's tumor).

In one aspect, the methods of treatment comprise administering to an individual a composition comprising an antigen-binding compound that binds MICA in a therapeutically effective amount. A therapeutically effective amount may be for example an amount sufficient to cause the depletion, or an increase in the depletion, of MICA cells in vivo, or an increase in the frequency of activated, reactive, cytotoxic and/or IFNγ-production of NKG2D+ effector cells (e.g., NK cells) towards MICA-expressing tumor cells. A therapeutically effective amount may be for example an amount sufficient to overcome or reduce M2 macrophage-mediated suppression of NK cell and/or T cell activity. In another example, a therapeutically effective amount may be for example an amount sufficient to overcome or reduce myeloid-derived suppression cell (MDSC)-mediated suppression of NK cell and/or T cell activity, or an amount sufficient to eliminate myeloid-derived suppression cells (MDSC) and/or M2 macrophages, e.g., in a tumor tissue.

The methods are utilized advantageously for the treatment of cancers and other proliferative diseases including, but not limited to, carcinoma, including that of the bladder, breast, colon, kidney, head and neck (e.g. head and neck squamous cell carcinoma), liver, lung, ovary, prostate, pancreas, stomach, cervix, thyroid and skin, including squamous cell carcinoma; hematopoietic tumors of lymphoid lineage, including leukemia, acute lymphocytic leukemia, acute lymphoblastic leukemia, B-cell lymphoma, T-cell lymphoma, Hodgkins lymphoma, non-Hodgkins lymphoma, hairy cell lymphoma and Burketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including neuroblastoma and glioma; tumors of the central and peripheral nervous system, including astrocytoma, neuroblastoma, glioma, and schwannomas; tumors of mesenchymal origin, including fibrosarcoma, rhabdomyoscaroma, and osteosarcoma; and other tumors, including melanoma, xeroderma pigmentosum, keratoacanthoma, seminoma, thyroid follicular cancer and teratocarcinoma. Other exemplary disorders that can be treated include hematopoietic tumors of lymphoid lineage, for example T-cell and B-cell tumors, including but not limited to T-cell disorders such as T-prolymphocytic leukemia (T-PLL), including of the small cell and cerebriform cell type; large granular lymphocyte leukemia (LGL) optionally of the T-cell type; Sezary syndrome (SS); Adult T-cell leukemia lymphoma (ATLL); a/d T-NHL hepatosplenic lymphoma; peripheral/post-thymic T cell lymphoma (pleomorphic and immunoblastic subtypes); angio immunoblastic T-cell lymphoma; angiocentric (nasal) T-cell lymphoma; anaplastic (Ki 1+) large cell lymphoma; intestinal T-cell lymphoma; T-lymphoblastic; and lymphoma/leukaemia (T-Lbly/T-ALL).

In some embodiments, prior to the administration of the anti-MICA antibody or composition, the presence of MICA on cells (e.g., tumor cells) of the patient will be assessed, e.g., to determine the relative level and activity of MICA-positive cells in the patient as well as to confirm the binding efficacy of the antibodies to the cells of the patient. A patient whose tumor cells express MICA can then be treated with an anti-MICA antibody or composition. This can be accomplished by obtaining a sample of sPBLs or tumor cells from the site of the disorder, and testing e.g., using immunoassays, to determine the relative prominence of MICA and optionally further other markers on the cells. Other methods can also be used to detect expression of MICA and other genes, such as RNA-based methods, e.g., RT-PCR or Northern blotting. Optionally, soluble MICA is used as a marker for the presence of tumor cells expressing MICA at their surface. In one embodiment, a serum sample is obtained from an individual and the presence of soluble MICA is assessed, wherein a detection of soluble MICA in serum from an individual indicates that the individual has tumor comprising tumor cells that express MICA at their surface (membrane bound MICA).

In one embodiment, where it is sought to inhibit the activity or growth of, or deplete, a patient's MICA-positive cells, the ability of the anti-MICA antibody to inhibit proliferation of or deplete a patient's MICA-positive cells is assessed. If the MICA-positive cells are depleted by the anti-MICA antibody or composition, the patient is determined to be responsive to therapy with an anti-MICA antibody or composition, and optionally the patient is treated with an anti-MICA antibody or composition.

The treatment may involve multiple rounds of anti-MICA antibody or compound administration. For example, following an initial round of administration, the level and/or activity of MICA-expressing cells (e.g., by detecting presence and/or levels of soluble MICA in serum of an individual), in an individual will generally be re-measured, and, if still elevated, an additional round of administration can be performed. In this way, multiple rounds of MICA detection and antibody or compound administration can be performed, e.g., until the disorder is brought under control.

In some embodiments, the method may comprise the additional step of administering to said patient an appropriate additional (second) therapeutic agent selected from an immunomodulatory agent, a hormonal agent, a chemotherapeutic agent, or a second antibody (e.g., a depleting antibody) that binds to a polypeptide present on a MICA-expressing cell. Such additional agents can be administered to said patient as a single dosage form together with said antibody, or as a separate dosage form. The dosage of the antibody (or antibody and the dosage of the additional therapeutic agent collectively) are sufficient to detectably induce, promote, and/or enhance a therapeutic response in the patient. Where administered separately, the antibody, fragment, or derivative and the additional therapeutic agent are desirably administered under conditions (e.g., with respect to timing, number of doses, etc.) that result in a detectable combined therapeutic benefit to the patient.

For tumor (e.g., solid tumor) treatment, for example, the administration of an anti-MICA antibody composition of the disclosure may be used in combination with classical approaches, such as surgery, radiotherapy, chemotherapy, and the like. The disclosure therefore provides combined therapies in which the present antibodies are used simultaneously with, before, or after surgery or radiation treatment; or are administered to patients with, before, or after conventional chemotherapeutic, radiotherapeutic or anti-angiogenic agents, or targeted immunotoxins or coaguligands.

Exemplary anti-cancer anti-angiogenic agents inhibit signaling by a receptor tyrosine kinase including but not limited to FGFR (fibroblast growth factor receptor, FGF-1,2), PDGFR (platelet derived growth factor receptor), angiopoietins receptors (Ang-1,2), HGFR (hepatocytary growth factor receptor), ephrines receptor (Eph), VEGFR1, VEGFR-2,3 PDGFR-α, PDGFR-β, CSF-1R, MET, Flt-3, c-Kit, bcr/abl, p38 alpha and FGFR-1. Further anti-angiogenic agents may include agents that inhibit one or more of the various regulators of VEGF expression and production, such as EGFR, flt-1, KDR, HER-2, COX-2, or HIF-1α. Another preferred class of agents includes IMiD (immunomodulatory drugs), analogs derived from thalidomide that have a wide range of effects, including both immune and non-immune related effects. Representatives of the IMiD class include CC-5013 (lenalidomide, Revlimid™), CC-4047 (Actimid™), and ENMD-0995. Another class of anti-angiogenic agent includes cilengitide (EMD 121974, integrin inhibitor), metalloproteinases (MPP) such as marinastat (BB-251). Another class of anti-angiogenic agents includes farnesylation inhibitors such as Ionafarnib (Sarasar™), tipifarnib (Zarnestra™). Other anti-angiogenic agents can also be suitable such as Bevacuzimab (mAb, inhibiting VEGF-A, Genentech); IMC-1121B (mAb, inhibiting VEGFR-2, ImClone Systems); CDP-791 (Pegylated DiFab, VEGFR-2, Celltech); 2C3 (mAb, VEGF-A, Peregrine Pharmaceuticals); VEGF-trap (Soluble hybrid receptor VEGF-A, PIGF (placenta growth factor) Aventis/Regeneron). Another preferred class of agents includes the tyrosine kinase inhibitor (TKI) class, including, e.g., PTK-787 (TKI, VEGFR-1,-2, Vatalanib, Novartis); AEE788 (TKI, VEGFR-2 and EGFR, Novartis); ZD6474 (TKI, VEGFR-1,-2,-3, EGFR, Zactima, AstraZeneca); AZD2171 (TKI, VEGFR-1,-2, AstraZeneca); SU11248 (TKI, VEGFR-1,-2, PDGFR, Sunitinib, Pfizer); AG13925 (TKI, VEGFR-1,-2, Pfizer); AG013736 (TKI, VEGFR-1,-2, Pfizer); CEP-7055 (TKI, VEGFR-1,-2,-3, Cephalon); CP-547,632 (TKI, VEGFR-1,-2, Pfizer); GW786024 (TKI, VEGFR-1,-2,-3, GlaxoSmithKline); GW786034 (TKI, VEGFR-1,-2,-3, GlaxoSmithKline); sorafenib (TKI, Bay 43-9006, VEGFR-1,-2, PDGFR Bayer/Onyx); SU4312 (TKI, VEGFR, PDGFR, Pfizer), AMG706 (TKI, VEGFR-1,-2,-3, Amgen), XL647 (TKI, EGFR, HER2, VEGFR, ErbB4, Exelixis), XL999 (TKI, FGFR, VEGFR, PDGFR, Flt-3, Exelixis), PKC412 (TKI, KIT, PDGFR, PKC, FLT3, VEGFR-2, Novartis), AEE788 (TKI, EGFR, HER2, VEGFR, Novartis), OSI-930 (TKI, c-kit, VEGFR, OSI Pharmaceuticals), OSI-817 (TKI, c-kit, VEGFR, OSI Pharmaceuticals), DMPQ (TKI, ERGF, PDGFR, erbB2, p56, pkA, pkC), MLN518 (TKI, FLT3, PDGFR, c-KIT, CT53518, Millennium Pharmaceuticals), lestaurinib (TKI, FLT3, CEP-701, Cephalon), ZD1839 (TKI, EGFR, gefitinib, Iressa, AstraZeneca), OSI-774 (TKI, EGFR, Erlotininb, Tarceva, OSI Pharmaceuticals), lapatinib (TKI, ErbB-2, EGFR, GD-2016, Tykerb, GlaxoSmithKline). Examples of tyrosine kinase inhibitors that inhibit one or more receptor tyrosine kinases selected from the group consisting of VEGFR-1, VEGFR-2, VEGFR-3, PDGFR-α, β, Flt-3, c-Kit, p38 alpha, MET, c-RAF, b-RAF, bcr/abl and FGFR-1.

In one embodiment, the second agent is a natural ligand of an effector cell (e.g., NK cell) activating receptor or an antibody that binds and activates an NK cell activating receptor other than NKG2D. In one embodiment the agent is an agent that increases the presence of a natural ligand of an NK cell activating receptor other than NKG2D on the surface of a target cell (e.g., infected cells, tumor cells, pro-inflammatory cells). NK cell activating receptors include, for example, natural cytotoxicity receptors such as NKp30, NKp46, NKp44 or activating KIR receptors (KIR2DS receptors, KIR2DS2, KIR2DS4). As used herein, the term “activating NK receptor” refers to any molecule on the surface of NK cells that, when stimulated, causes a measurable increase in any property or activity known in the art as associated with NK activity, such as cytokine (for example IFN-γ and TNF-α production, increases in intracellular free calcium levels, the ability to target cells in a redirected killing assay as described, e.g., elsewhere in the present specification, or the ability to stimulate NK cell proliferation. The term “activating NK receptor” includes but is not limited to activating forms or KIR proteins (for example KIR2DS proteins), NKp30, NKp46, NKp44, NKG2D, IL-2R, IL-12R, IL-15R, IL-18R and IL-21R.

In one embodiment, the anti-cancer agent is a chemotherapeutic agent or radiation that upregulates expression of NKG2D ligands on the surface of tumor cells. This includes well known chemotherapies including ionizing and UV radiation, inhibitors of DNA replication, inhibitors of DNA polymerase, chromatin modifying treatments, as well as apoptosis inducing agents such as HDAC inhibitors trichostatin A and valproic acid. Preferred therapies are those that activate the DNA damage response pathway, for example those that activate the ATM (ataxia telangiectasia, mutated) or ATR (ATM- and Rad3-related) protein kinases, or CHK1, or yet further CHK2 or p53. Examples of the latter include ionizing radiation, inhibitors of DNA replication, DNA polymerase inhibitors and chromatic modifying agents or treatment including HDAC inhibitors. Compositions that upregulate NKG2D ligands are further described in Gasser et al (2005) Nature 436(7054):1186-90. NKG2D is an activating receptor that interacts with the MHC class I-related MICA and MICB glycoproteins, among other ligands. MICA and MICB (Bauer et al. (1999) Science 285:727-729, the disclosure of which is incorporated herein by reference) have no role in antigen presentation, are generally only found in intestinal epithelium, and can be stress-induced in permissive types of cells by viral and bacterial infections, malignant transformation, and proliferation. NKG2D is a C-type lectin-like activating receptor that signals through the associated DAP10 adaptor protein, which is similar to CD28. It is expressed on most natural killer (NK) cells, NKT cells, γδ T cells CD8 T cells, and T cells, but not, in general, on CD4 T cells. Other NKG2D ligands include ULBP proteins, e.g., ULBP-1, -2, -3, -4, -5 and -6, originally identified as ligands for the human cytomegalovirus glycoprotein UL16 (Cosman et al, (2001) Immunity 14: 123-133, and Raulet et al, (2013) Ann Review Immunology 31:413-41, the disclosures of which are incorporated herein by reference).

Further anti-cancer agents include alkylating agents, cytotoxic antibiotics such as topoisomerase I inhibitors, topoisomerase II inhibitors, plant derivatives, RNA/DNA antimetabolites, and antimitotic agents. Preferred examples may include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, taxol, gemcitabine, navelbine, transplatinum, 5-fluorouracil, vincristin, vinblastin and methotrexate, or any analog or derivative variant of the foregoing.

In the treatment methods, the anti-MICA antibody of the disclosure and the second therapeutic agent can be administered separately, together or sequentially, or in a cocktail. In some embodiments, the anti-MICA antibody is administered prior to the administration of the second therapeutic agent. For example, anti-MICA antibody can be administered approximately 0 to 30 days prior to the administration of the second therapeutic agent. In some embodiments, an anti-MICA antibody is administered from about 30 minutes to about 2 weeks, from about 30 minutes to about 1 week, from about 1 hour to about 2 hours, from about 2 hours to about 4 hours, from about 4 hours to about 6 hours, from about 6 hours to about 8 hours, from about 8 hours to 1 day, or from about 1 to 5 days prior to the administration of the second therapeutic agent. In some embodiments, an anti-MICA antibody is administered concurrently with the administration of the therapeutic agents. In some embodiments, an anti-MICA antibody is administered after the administration of the second therapeutic agent. For example, an anti-MICA antibody can be administered approximately 0 to 30 days after the administration of the second therapeutic agent. In some embodiments, an anti-MICA antibody is administered from about 30 minutes to about 2 weeks, from about 30 minutes to about 1 week, from about 1 hour to about 2 hours, from about 2 hours to about 4 hours, from about 4 hours to about 6 hours, from about 6 hours to about 8 hours, from about 8 hours to 1 day, or from about 1 to 5 days after the administration of the second therapeutic agent.

The anti-MICA antibody of the disclosure can be included in kits. The kits may optionally further contain any number of antibodies and/or other compounds, e.g., 1, 2, 3, 4, or any other number of anti-MICA antibodies and/or other compounds. It will be appreciated that this description of the contents of the kits is not limiting in any way. For example, the kit may contain other types of therapeutic or diagnostic agents. The kits may also include instructions for using the antibodies and/or agents, e.g., detailing the herein-described methods.

### EXAMPLES

### Example 1: Production of Modified Anti-MICA Antibodies

The antibodies having the VH and Vk variable regions shown below were produced as human IgG1 antibodies with human frameworks and murine Kabat CDRs as described herein. Briefly, the VH and Vk sequences of each antibody were cloned into vectors containing the hulgG1-derived constant domains and the huCk constant domain respectively. The two obtained vectors were co-transfected into a CHO cell line. The established pool of cell was used to produce the antibody in the CHO medium. The antibody was then purified using protein A.

3D models based on different human VH gene segments were superimposed and all amino acid differences were scrutinized one by one.

In order to investigate whether introduction of a salt bridge that could impact positioning of the CDR1 of the light chain could in turn affect binding to MICA, residue F71 (Abm numbering) in the light chain was substituted by a tyrosine (Y) within the tipeptide DFT 4 DYT (the F71Y substitution). Substitution of F by Y at residue 71 just below the CDR_L1 loop might form H-bonds with CDR residues.

Additionally, in a further variant light chain a double substitution (the S70D/F71Y substitution) was made to replace D70 by S70 (Abnum numbering), yet without substitution T72; the resulting tripeptide at Abnum resides 70-72 was thus changed from SYT to DFT. Finally, a substitution at residue 83 (Abm numbering) was made in a variant of the light chain into which was introduced the single F71Y substitution. The V83 radical is exposed at the VL/CK interface whereas the F83 radical is buried inside de VL domain hydrophobic cavity.

In the heavy chain, four variant chains were constructed that all had substitutions at Abnum residue 30 (S30T substitution, framework 1) and at Abnum residue 71 (V71R substitution, framework 3), wherein a residue present in murine antibodies was substituted for the residue in the human sequence. Residue 30 is a CDR flanking residue which might face the antigen. Residue 71 takes a critical position just below the top of the CDR_H2 loop and form h-bonds with CDR_H2 residues.

In three of these variant chains (chains 2, 3 and 4) a further substitution was introduced in framework 2 at Abnum residue 48. Isoleucine was substituted by a methionine (148M substitution). M48 is a Vernier zone residue which is located just below the CDR_H2. While it does not form any h-bond with adjacent residue in murine antibody, Vernier zone residues might be critical for CDR positioning.

Additionally, in two variant heavy chains (chains 3 and 4), a further framework 3 substitution was made at Abnum residue 72c (K72cE substitution). K72c forms a H-bond with Q74 in a murine framework. E72c and K72c adopt divergent conformations mainly because of the h-bond formed between K72c and Q74. The possible salt bridge was therefore removed at residue 72-74.

Finally, in one variant heavy chain (chain 4), a further framework 3 substitution was made at Abnum residue 67 (V671 substitution). 167 is a Vernier zone residue located below the CDR_H2.

In the light chain, Abnum residues 70, 71 and 83 correspond respectively to residues at positions 71, 72 and 84 of the sequence listing (e.g., SEQ ID NO: 7 or 9). In the heavy chain, Abnum residues 30, 48, 67, 71, 72c and 74 correspond respectively to residues at positions 30, 49, 68, 72, 76 and 78 of the sequence listing (e.g., SEQ ID NO: 6 or 8).

The amino acid sequences of respective heavy and light chain variable regions are shown in the Table 2 below.

TABLE 2

| Antibody | SEQ | |
|---|---|---|
| domain | ID | Amino acid sequence |
| mAb1 | 6 | QVQLQESGPGLVKPSETLSLTCTVSGYSITSDYAWNWIRQPPGKGLEWIGFVSYSGTTKY |
| VH | | NPSLKSRVTISRDTSKNQFSLKLSSVTAADTAVYYCARGYGFDYWGQGTTVTVSS |
| mAb1 | 7 | EIVLTQSPATLSLSPGERATLSCSATSSISSIYFHWYQQKPGQAPRLLIYRTSNLASGIP |
| VL | | ARFSGSGSGTDYTLTISSLEPEDFAVYYCQQGTTIPFTFGQGTKLEIK |
| mAb2 | 8 | QVQLQESGPGLVKPSETLSLTCTVSGYSITSDYAWNWIRQPPGKGLEWIGFVSYSGTTKY |
| VH | | NPSLKSRVTISRDTSKNQFSLKLSSVTAADTAVYYCARGYGFDYWGQGTTVTVSS |
| mAb2 | 9 | EIVLTQSPATLSLSPGERATLSCSATSSISSIYFHWYQQKPGQAPRLLIYRTSNLASGIP |
| VL | | ARFSGSGSGTSYTLTISSLEPEDFAVYYCQQGTTIPFTFGQGTKLEIK |
| mAb3 | 10 | QVQLQESGPGLVKPSETLSLTCTVSGYSITSDYAWNWIRQPPGKGLEWIGFVSYSGTTKY |
| VH | | NPSLKSRVTISRDTSKNQFSLKLSSVTAADTAVYYCARGYGFDYWGQGTTVTVSS |
| mAb3 | 11 | EIVLTQSPATLSLSPGERATLSCSATSSISSIYFHWYQQKPGQAPRLLIYRTSNLASGIP |
| VL | | ARFSGSGSGTDYTLTISSLEPEDVAVYYCQQGTTIPFTFGQGTKLEIK |
| mAb4 | 12 | QVQLQESGPGLVKPSETLSLTCTVSGYSITSDYAWNWIRQPPGKGLEWMGEVSYSGTTKY |
| VH | | NPSLKSRVTISRDTSKNQFSLKLSSVTAADTAVYYCARGYGFDYWGQGTTVTVSS |
| mAb4 | 13 | EIVLTQSPATLSLSPGERATLSCSATSSISSIYFHWYQQKPGQAPRLLIYRTSNLASGIP |
| VL | | ARFSGSGSGTDYTLTISSLEPEDFAVYYCQQGTTIPFTFGQGTKLEIK |
| mAb5 | 14 | QVQLQESGPGLVKPSETLSLTCTVSGYSITSDYAWNWIRQPPGKGLEWMGEVSYSGTTKY |
| VH | | NPSLKSRVTISRDTSKNQFSLKLSSVTAADTAVYYCARGYGFDYWGQGTTVTVSS |
| mAb5 | 15 | EIVLTQSPATLSLSPGERATLSCSATSSISSIYFHWYQQKPGQAPRLLIYRTSNLASGIP |
| VL | | ARFSGSGSGTSYTLTISSLEPEDFAVYYCQQGTTIPFTFGQGTKLEIK |
| mAb6 | 16 | QVQLQESGPGLVKPSETLSLTCTVSGYSITSDYAWNWIRQPPGKGLEWMGEVSYSGTTKY |
| VH | | NPSLKSRVTISRDTSKNQFSLKLSSVTAADTAVYYCARGYGFDYWGQGTTVTVSS |
| mAb6 | 17 | EIVLTQSPATLSLSPGERATLSCSATSSISSIYFHWYQQKPGQAPRLLIYRTSNLASGIP |
| VL | | ARFSGSGSGTDYTLTISSLEPEDVAVYYCQQGTTIPFTFGQGTKLEIK |
| mAb7 | 18 | QVQLQESGPGLVKPSETLSLTCTVSGYSITSDYAWNWIRQPPGKGLEWMGEVSYSGTTKY |
| VH | | NPSLKSRVTISRDTSENQFSLKLSSVTAADTAVYYCARGYGFDYWGQGTTVTVSS |
| mAb7 | 19 | EIVLTQSPATLSLSPGERATLSCSATSSISSIYFHWYQQKPGQAPRLLIYRTSNLASGIP |
| VL | | ARFSGSGSGTDYTLTISSLEPEDFAVYYCQQGTTIPFTFGQGTKLEIK |
| mAb8 | 20 | QVQLQESGPGLVKPSETLSLTCTVSGYSITSDYAWNWIRQPPGKGLEWMGEVSYSGTTKY |
| VH | | NPSLKSRVTISRDTSENQFSLKLSSVTAADTAVYYCARGYGFDYWGQGTTVTVSS |
| mAb8 | 21 | EIVLTQSPATLSLSPGERATLSCSATSSISSIYFHWYQQKPGQAPRLLIYRTSNLASGIP |
| VL | | ARFSGSGSGTSYTLTISSLEPEDFAVYYCQQGTTIPFTFGQGTKLEIK |
| mAb9 | 22 | QVQLQESGPGLVKPSETLSLTCTVSGYSITSDYAWNWIRQPPGKGLEWMGEVSYSGTTKY |
| VH | | NPSLKSRVTISRDTSENQFSLKLSSVTAADTAVYYCARGYGFDYWGQGTTVTVSS |
| mAb9 | 23 | EIVLTQSPATLSLSPGERATLSCSATSSISSIYFHWYQQKPGQAPRLLIYRTSNLASGIP |
| VL | | ARFSGSGSGTDYTLTISSLEPEDVAVYYCQQGTTIPFTFGQGTKLEIK |
| mAb10 | 24 | QVQLQESGPGLVKPSETLSLTCTVSGYSITSDYAWNWIRQPPGKGLEWMGEVSYSGTTKY |
| VH | | NPSLKSRITISRDTSENQFSLKLSSVTAADTAVYYCARGYGFDYWGQGTTVTVSS |
| mAb10 | 25 | EIVLTQSPATLSLSPGERATLSCSATSSISSIYFHWYQQKPGQAPRLLIYRTSNLASGIP |
| VL | | ARFSGSGSGTDYTLTISSLEPEDFAVYYCQQGTTIPFTFGQGTKLEIK |
| mAb11 | 26 | QVQLQESGPGLVKPSETLSLTCTVSGYSITSDYAWNWIRQPPGKGLEWMGEVSYSGTTKY |
| VH | | NPSLKSRITISRDTSENQFSLKLSSVTAADTAVYYCARGYGFDYWGQGTTVTVSS |
| mAb11 | 27 | EIVLTQSPATLSLSPGERATLSCSATSSISSIYFHWYQQKPGQAPRLLIYRTSNLASGIP |
| VL | | ARFSGSGSGTSYTLTISSLEPEDFAVYYCQQGTTIPFTFGQGTKLEIK |
| mAb12 | 28 | QVQLQESGPGLVKPSETLSLTCTVSGYSITSDYAWNWIRQPPGKGLEWMGEVSYSGTTKY |
| VH | | NPSLKSRITISRDTSENQFSLKLSSVTAADTAVYYCARGYGFDYWGQGTTVTVSS |
| mAb12 | 29 | EIVLTQSPATLSLSPGERATLSCSATSSISSIYFHWYQQKPGQAPRLLIYRTSNLASGIP |
| VL | | ARFSGSGSGTDYTLTISSLEPEDVAVYYCQQGTTIPFTFGQGTKLEIK |

### Example 2: Binding to MICA Alleles

The binding of the antibodies in Table 2 of Example 1 were tested for binding to MICA-expressing C1R transfectant cells (ATCC reference CRL-1993™) transfected with RSV.5neo vectors (GenBank (NCBI) under Accession number M83237), as described in Salih et al. (2003) Blood 102(4): 1389-91396, referred to as C1R-MICA*001, C1R-MICA*004, C1R-MICA*007 and C1R-MICA*008. Binding was analyzed by flow cytometry.

Flow Cytometry.

Cells were harvested and stained in PBS 1×/BSA 0.2%/EDTA 2 mM buffer during 30 minutes at 4° C. using a dose-range of the anti-MICA mAbs. After two washes in staining buffer, cells were stained for 30 min at 4° C. with mouse anti-human IgG1-PE monoclonal antibodies (1/11). After two washes, stainings were acquired on a BD FACS Canto II and analyzed using the FlowJo software.

Results.

Each of the antibodies in Table 3 showed high affinity binding across all of the MICA alleles. Surprisingly, however, mAb1 and mAb2 showed a particularly strong improvement in binding affinity for MICA alleles *01, *04 and *07. Affinity for MICA*01 was improved by more than 2-fold (2.4-fold for mAb1 and about 3-fold for mAb2) compared to the parental antibody having the murine parental VH and VL and sharing the same human constant regions as mAbs1-12 and other human framework antibodies. mAb3 also showed improved binding affinity compared to the parental chimeric antibody, although to a lesser degree than mAbs1 and 2. Results are shown in Table 3 below.

mAbs1, 2 and 3 all share a heavy chain in which a lysine (K) is present at position 72c (Abnum). A lysine acid at this position can introduce a salt bridge between residues 72c and 74. The salt bridge was not introduced in the other heavy chains used in various other mAbs which had a glutamic acid (E) at residue 72c in the VH. The heavy chain of mAbs1, 2 and 3 further has an isoleucine at position 48. mAbs1 and 2 made use of light chains in which a tyrosine (Y) replaces a phenylalanine at residue 71 (Abm numbering) just below the CDRL1 loop so as to form a possible salt bridge (H-bonds) with CDR residues, thereby possibly changing the positioning of the CDR. mAb3 differs from mAbs1 and 2 in that a phenylalanine (F) is present at position 83 in the VL in mAbs1 and 2 while mAb3 has a valine (V) at position 83 in the light chain (Abnum numbering).

TABLE 3
EC₅₀ values in μg/ml of indicated anti-MICA antibodies on
C1R transfectant cells

| | C1R- | C1R- | C1R- | C1R- |
|---|---|---|---|---|
| Antibody | MICA*01 | MICA*04 | MICA*07 | MICA*08 |
| Parental antibody | 0.4392 | 0.0618 | 0.0698 | 0.0716 |
| mAb1 | 0.1841 | 0.0417 | 0.0330 | 0.0612 |
| mAb2 | 0.1508 | 0.0381 | 0.0502 | 0.0699 |
| mAb3 | 0.2645 | 0.0608 | 0.0612 | 0.0822 |
| mAb4 | 0.3164 | 0.0630 | 0.0875 | 0.0859 |
| mAb5 | 0.3177 | 0.0762 | 0.0493 | 0.0958 |
| mAb6 | 0.4071 | 0.0838 | 0.0718 | 0.0953 |
| mAb7 | 0.2880 | 0.0616 | 0.0668 | 0.0871 |
| mAb8 | 0.1906 | 0.0481 | 0.0462 | 0.0551 |
| mAb9 | 0.3178 | 0.0438 | 0.0519 | 0.0515 |
| mAb10 | 0.3071 | 0.0631 | 0.0498 | 0.1443 |
| mAb11 | 0.3196 | 0.0614 | 0.0513 | 0.0789 |
| mAb12 | 0.4534 | 0.0672 | 0.0860 | 0.0642 |

### Example 3—Antibodies are Able to Kill MICA Expressing Targets Via ADCC

mAbs were tested for their ability to mediate ADCC towards C1R tumor cells transfected with MICA*008 (C1R-MICA*008) or MICA*001 (C1R-MICA*001).

Briefly, the cytolytic activity of human NK cell line KHYG-1 transfected with human CD16 (F isoform) was assessed in a classical 4-hour ⁵¹Cr-release assay in 96 well plates V from (Greiner). Briefly, C1R-MICA*008 cells were labelled with ⁵¹Cr (100 μCi (3.7 MBq)/1×106 cells), then mixed with KHYG-transfected with hCD16F (to bind human IgG1) at an effector/target ratio equal to 10, in the presence of antibody at indicated concentrations. After brief centrifugation and 4 hours of incubation at 37° C., 50 μL supernatant were removed, and the 51Cr release was measured with a TopCount NXT beta detector (PerkinElmer Life Sciences, Boston, Mass.). All experimental groups were analyzed in triplicate, and the percentage of specific lysis was determined as follows: 100×(mean cpm experimental release−mean cpm spontaneous release)/(mean cpm total release−mean cpm spontaneous release). Percentage of total release obtained by lysis of target cells with 2% Triton X100 (Sigma).

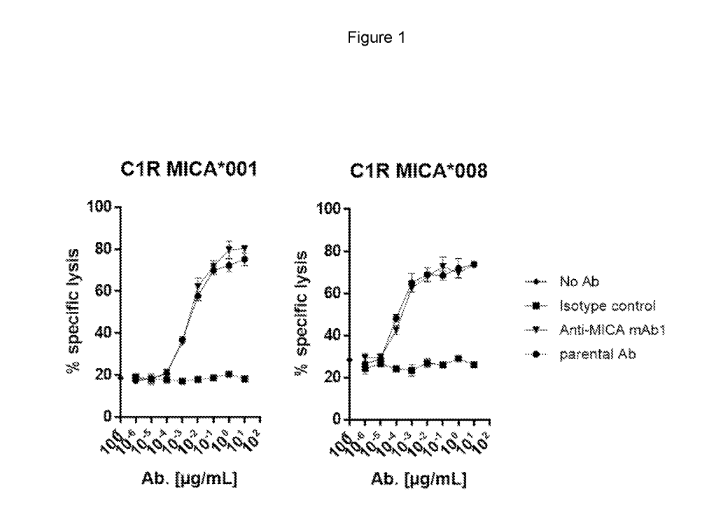

Results for mAb1 are shown in FIG. 1. mAb1 and the chimeric parental antibody each induced specific lysis of C1R-MICA*008 and *001 cells by human KHYG-1 hCD16F NK cell line compared to negative controls (Human IgG1 isotype control antibody), thereby showing that these antibodies induce ADCC toward MICA*008- and *001-expressing target cells. The extent of target cell lysis is correlated to antibody binding to the cell (FIG. 1); mAb1 induced somewhat greater specific lysis of *001 cells than the chimeric parental antibody.

### Example 4—Anti-MICA Antibody Overcomes M2 Macrophage-Mediated Suppression of NK Cell Activity

NK cells were incubated 24 hours with autologous in vitro monocyte-derived M1 or M2 macrophages. Then, culture supernatants containing non-adherent NK cells were incubated with LCL-721.221 cells (EBV-transfected B cell line) transfected with MICA*001 (LCL-721.221-MICA*001 cells) for an additional 24 hours. The activation marker CD137 on NK was measured by flow cytometry. Anti-MICA antibody mAb1 or isotype control (IC) were used at 10 μg/mL.

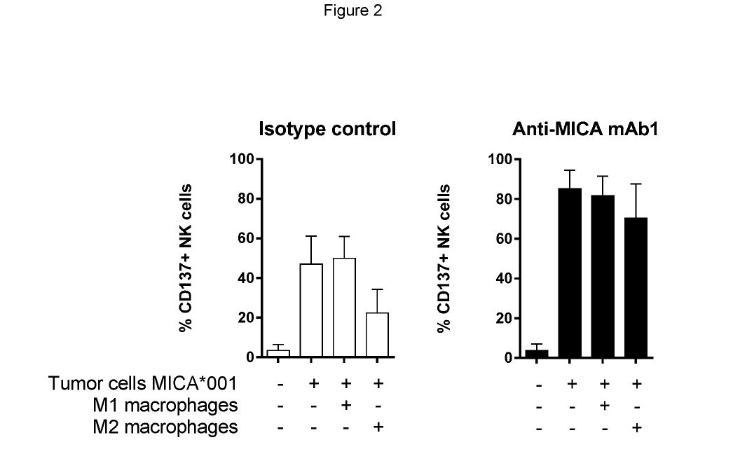

Results are shown in FIG. 2. Mean+/−SD, n=4-7 independent healthy donors. Anti-MICA mAb1 caused a strong increase in NK cell activation towards the 721.221-MICA*001 tumor cells, including tumor cells with or without M1 or M2 macrophages. The incubation of tumor cells and NK cells with M2 macrophages did not cause a substantial decrease in NK cell activation in the presence of mAb1. In contrast, in isotype control, not only was NK activation generally far lower, but incubation of tumor cells and NK cells with M2 macrophages caused a strong decrease in NK activation.

### Example 5—In Vivo Efficacy of Anti-MICA Antibodies in Murine Raji Tumor Model

Part 1: Intravenous Administration, Single Administration

NOD-SCID mice were engrafted intravenously (i.v.) with Raji human Burkitt's lymphoma cells transfected with MICA*001 (Raji-MICA*001 cells) and treated the same day with a single injection of anti-MICA mAb1 at 1 μg, 10 μg, 50 μg or 100 μg or isotype control (IC) at indicated doses (μg/mouse, i.v.).

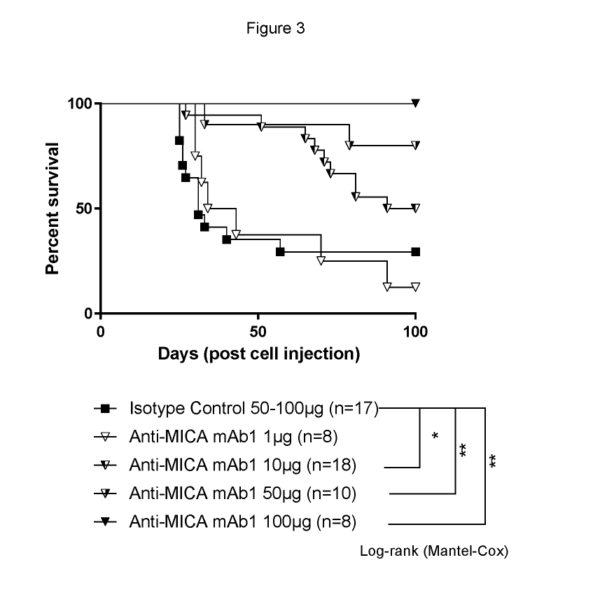

Results are shown in FIG. 3. While few mice receiving isotype control or 1 μg anti-MICA antibody mAb1 did not survive at 100 days post injection, significantly improved survival was observed in mice receiving at least 10 μg of anti-MICA antibody. At the 100 μg dose, anti-MICA antibody mAb1 achieved survival in all mice at 100 days. Log rank (Mantel-Cox) test, 10 μg p=0.0303, 50 μg p=0.0081, 100 μg p=0.0024.

Part 2: Subcutaneous, Repeat Administration NOD-SCID mice (n=12/group) were engrafted s.c. with Raji-MICA*001 cells. Mice were randomized at day 10 (tumor volume ˜120 mm³) and were then treated with anti-MICA antibody or isotype control (IC) (250 μg/mouse, i.v., twice a week for 3wks).

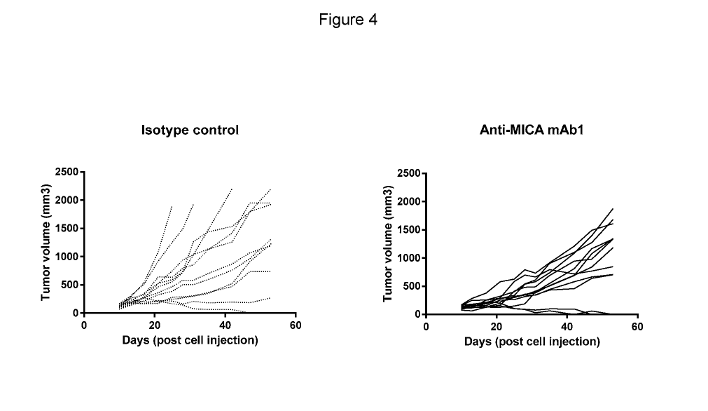

Results are shown in FIG. 4. The left hand panel shows mice receiving isotype control, and the right hand panel shows mice receiving anti-MICA antibody mAb1. Individual tumor volumes are shown. CR=complete response. Treatment with anti-MICA antibody mAb1 caused a decrease in tumor volume. Furthermore, 17% of mice treated with mAb1 experienced a complete response compared to 8% of mice receiving isotype control.

### Example 6—In Vivo Efficacy of Anti-MICA Antibodies in Murine A549 Tumor Model

NOD-SCID mice (n=7/group) were injected intraperitoneally (i.p.) with A549 cells (human lung carcinoma; ATCC Ref. CCL-185) and treated with a single injection of anti-MICA antibody mAb11 or isotype control (IC) (10 μg/mouse, i.v.). A549 cell number in peritoneal cavity lavage (PCL) was assessed 24 h after treatment.

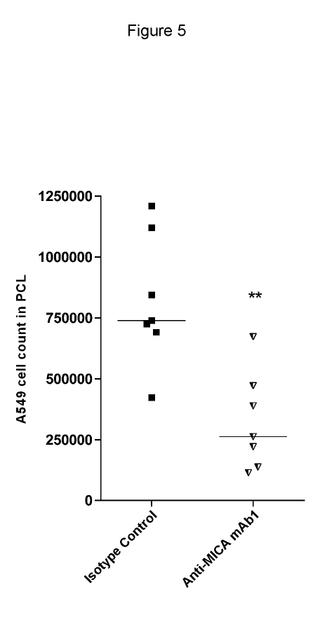

Results are shown in FIG. 5. Mice treated with anti-MICA antibody mAb1 exhibited a decreased tumor cell count compared to mice treated with isotype control. Individual mice and median are represented. Mann-Whitney comparison p=0.0023.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The use of the terms “a” and “an” and “the” and similar references are to be construed to cover both the singular and the plural, unless otherwise indicated herein or clearly contradicted by context.

Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

The description herein of any aspect or embodiment herein using terms such as “comprising”, “having,” “including,” or “containing” with reference to an element or elements is intended to provide support for a similar aspect or embodiment herein that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

1. A monoclonal antibody or antibody fragment that binds a human MICA polypeptide, wherein the antibody or antibody fragment comprises:
(a) a heavy chain variable region comprising the amino acid sequence of SEQ ID NO: 6, and a light chain variable region (VL) comprising the amino acid sequence of SEQ ID NO: 7;
or
(b) a heavy chain variable region comprising an amino acid sequence of SEQ ID NO: 8 and a light chain variable region (VL) comprising an amino acid sequence of SEQ ID NO: 9.

2. The antibody of claim 1, wherein the antibody comprises a heavy chain variable region comprising the amino acid sequence of SEQ ID NO: 6, and a light chain variable region (VL) comprising the amino acid sequence of SEQ ID NO: 7.

3. The antibody of claim 1, wherein the antibody comprises heavy chain variable region comprising the amino acid sequence of SEQ ID NO: 8 and a light chain variable region (VL) comprising the amino acid sequence of SEQ ID NO: 9.

4. An antibody or antibody fragment that binds a human MICA polypeptide, wherein the antibody or antibody fragment comprises: a heavy chain variable region (VH) comprising an amino acid sequence at least 90%, 95% or 98% identical to the amino acid sequence of SEQ ID NO: 6, and a light chain variable region (VL) comprising an amino acid sequence at least 90%, 95% or 98% identical to the amino acid sequence of SEQ ID NO: 7, wherein the VH comprises a lysine (K) residue at position 72c and a glutamine (Q) residue at position 74 and the VL comprises a tyrosine (Y) at position 71, wherein numbering of residues is according to Abnum numbering).

5. The composition of claim 4, wherein the VL comprises a phenylalanine (F) at Abnum position 83.

6. The composition of claim 4, wherein the VH comprises a threonine (T) at Abnum position 30, a valine (V) at position 67 and an arginine (R) at position 71.

7. The composition of claim 4, wherein the VH comprises an isoleucine (I) at Abnum position 48.

8. The composition of claim 4, wherein the VH comprises a methionine (M) at Abnum position 48.

9. The composition of claim 4, wherein the VH human acceptor framework is from IGHV4-b and the J-segment is from IGHJ6.

10. The composition of claim 4, wherein the VL domain human acceptor framework is from IGKV3-11 and the J-segment is from IGKJ2.

11. The composition of claim 4, wherein the antibody comprises: a CDR-H1 comprising the amino acid sequence of SEQ ID NO: 30; a CDR-H2 comprising the amino acid sequence of SEQ ID NO: 31; a CDR-H3 comprising the amino acid sequence of SEQ ID NO: 32; a CDR-L1 comprising the amino acid sequence of SEQ ID NO: 33; a CDR-L2 comprising the amino acid sequence of SEQ ID NO:34; and a CDR-L3 comprising the amino acid sequence of SEQ ID NO: 35.

12. The composition of claim 1, wherein the antibody binds to a cell surface MICA polypeptide comprising an amino acid sequence of SEQ ID NO: 1, to a cell surface MICA polypeptide comprising an amino acid sequence of SEQ ID NO: 2, to a cell surface MICA polypeptide comprising an amino acid sequence of SEQ ID NO: 3, and to a cell surface MICA polypeptide comprising an amino acid sequence of SEQ ID NO: 4.

13. The composition of claim 4, wherein the antibody is characterized by an EC₅₀, as determined by flow cytometry, of no more than 1 μg/ml or optionally no more than 0.2 μg/ml, for binding to C1R cells made to express at their surface a MICA polypeptide comprising an amino acid sequence of SEQ ID NO: 1, to C1R cells made to express at their surface a MICA polypeptide comprising an amino acid sequence of SEQ ID NO: 2, to C1R cells made to express at their surface a MICA polypeptide comprising an amino acid sequence of SEQ ID NO: 3, and, to C1R cells made to express at their surface a MICA polypeptide comprising an amino acid sequence of SEQ ID NO: 4.

14. The composition of claim 1, wherein the antibody further binds to a cell surface MICB polypeptide comprising an amino acid sequence of SEQ ID NO: 36.

15. The composition of claim 1, wherein the antibody blocks the interaction of membrane-bound human MICA with NKG2D.

16. The composition of claim 1, wherein the VH is fused to a human heavy chain constant domain and the VL is fused to a human light chain constant region.

17. The composition of claim 1, wherein said antibody comprises a human heavy chain constant region that binds a human FcγIIIA receptor.

18. The composition of claim 1, wherein said antibody has a Kd of less than 10⁻⁹ M for bivalent binding to a MICA polypeptide, as determined by surface plasmon resonance.

19. The composition of claim 1, wherein said antibody is an antibody fragment selected from Fab, Fab′, Fab′-SH, F(ab′) 2, Fv, a diabody, a single-chain antibody fragment, or a multispecific antibody comprising fragments from different antibodies.

20. The composition of claim 1, wherein said antibody is conjugated or covalently bound to a toxic agent.

21. The composition of claim 1, wherein said antibody is conjugated or covalently bound to a detectable moiety.

22. A pharmaceutical composition comprising an antibody according to claim 1, and a pharmaceutically acceptable carrier.

23. A pharmaceutical composition comprising an antibody according to claim 4, and a pharmaceutically acceptable carrier.

24. A recombinant host cell producing the antibody of claim 1.

25. A method for the treatment or prevention of a cancer in a human patient in need thereof, the method comprising administering to said patient an effective amount of a composition of claim 22.

26. A method for identifying a MICA-expressing cell in a subject, the method comprising obtaining a biological sample from a subject, bringing said cells into contact with an antibody of claim 1 and assessing whether the antibody binds to MICA and/or MICB in the sample.

27. A method for identifying a MICA-expressing disease-related cell in a subject, the method comprising obtaining a biological sample from a subject comprising disease-related cells, bringing said disease-related cells or serum into contact with an antibody of claim 1 and assessing whether the antibody binds to disease-related cells or soluble MICA within serum, wherein a finding that the antibody binds to soluble MICA and/or disease-related cells indicates that the subject has a disease, that the subject harbors disease-related cells and/or that the disease-related cell expresses MICA.

28. A method for selecting a subject having a disease that responds to a treatment with an antibody of claim 1, the method comprising determining whether tumor cells in said subject express a MICA polypeptide, optionally whether tumor cells express elevated levels of a MICA polypeptide, the expression of a MICA polypeptide or elevated levels of a MICA polypeptide being indicative of a responder subject.

29. The method of claim 26, further comprising administering to a responder subject a pharmaceutical composition comprising a monoclonal antibody or antibody fragment that binds a human MICA polypeptide, wherein the antibody or antibody fragment comprises:
(a) a heavy chain variable region comprising the amino acid sequence of SEQ ID NO: 6, and a light chain variable region (VL) comprising the amino acid sequence of SEQ ID NO: 7; or
(b) a heavy chain variable region comprising an amino acid sequence of SEQ ID NO: 8 and a light chain variable region (VL) comprising an amino acid sequence of SEQ ID NO: 9.

30. The method of claim 25, wherein said cancer is a solid tumor characterized by MICA-expressing cells.

31. The method of claim 25, wherein said patient has cells bearing a MICA allele selected from the group consisting of: MICA*001, MICA*004, MICA*007 and MICA*008.

32. The method of claim 25, wherein the same administration regimen is used to treat patients whose cells express MICA*001, patients whose cells express MICA*004, patients whose cells express MICA*007 and patients whose cells express MICA*008.

33. The method of claim 25, wherein said method is free of a step prior to treatment of determining the identity of the particularly MICA alleles expressed in an individual.
